SEQUENCE LISTING (8 sequences)

SEQ ID NO: 1 - DNA, 1982 bp, Hydrilla verticillata
  CDS (6)..(1748)
caaacatgactgttgctaggtcggtcgttgcagtcaatctaagtggttcccttcaaaacagatacccagccagttcatcagtcagctgcttccttggcaaagagtacagatgcaacagtatgttaggattctgcggtagtggaaaattggcttttggcgcaaatgcaccctattctaagattgcagctaccaaaccaaagcccaaacttcgccctttgaaggtcaactgcatggatttcccaagacctgatatagataacactgctaatttcttggaagctgctgctctttcttcctcttttcgcaattcagcaagaccaagtaaacctcttcaagttgtaattgctggtgcaggtttggctggtctttcaacagcaaagtatctcgcagatgcagggcacatacccatactactggaggctagagatgtattgggtggcaaggtggcagcgtggaaagatgatgatggagactggtatgagacaggcctgcatatattttttggtgcatatcccaatgtgcagaatttatttggtgaacttggcataaatgatcgtctacaatggaaagagcattcaatgatttttgcgatgccaaacaagccaggggaatttagtcgctttgattttccagaagtacttcctgctccactaaatggaatatgggcaatccttaaaaacaatgaaatgctcacttggccagagaaagtgcaatttgctattggactactacctgcaatgattggggggcagccatatgttgaagctcaggatggcttaacagttcaagagtggatgagaaaacagggtgtgccggatcgagtcaatgacgaggttttcattgcaatgtcaaaggctcttaacttcataaaccctgatgaactttccatgcaatgcatcctgattgccttaaaccgtttccttcaggaaaagcatgggtcgaagatggcctttttagatggtaatccacctgaaagattatgtaagccaattgctgatcacatcgagtcattgggtggccaagtcatccttaattcccgaatacagaagattgagctgaatgcagacaaatccgtcaagcattttgtgctcaccaatggaaatataataacaggagatgcatatgtatttgcaacacctgttgatatcttgaagcttctgttacctgaagattggaaggagatttcatatttcaaaaaattggacaagttggttggcgtacctgtgataaatgtacacatatggtttgataggaagttgaagaacacatacgatcatcttcttttcagcaggagtccactgttgagcgtttatgcagacatgtctgttacatgcaaggaatactacaatccaaatcaatccatgcttgagctagtatttgcaccagcagagaaatggatttcatgcagtgacagtgaaatcattaacgcgactatgcaagagcttgctaaactctttccagatgagatttctgctgatcaaagcaaggccaaaattttgaaatatcatgttgtaaagaccccgaggtcagtttacaagacggtccctgattgtgaaccatgccggcctttgcaaagatctccaattgaagggttctacttggctggtgactacacaaagcagaagtatttggcctcaatggaaggtgccgtgttatctgggaagctatgtgctcaggcaattgtgcaggactgcagcttgttggcttctagggtacagaaaagcccacagacgttgacgattgcctgattcaggaaacttttatgcaggttcagtttgtagggggaatatttctggttttgtttcattcagatgtttttcttttagagcatatgtctttatagtaaaaactcccacctctttctcatgtatagctacatcagcaagcaaagggggtaagttgcaatttcaggacttgaacatggcctctgcacaggtaaagacagaatggacataaatgcaagcatggaatttacaatattc

SEQ ID NO: 2 - protein, 580 aa, Hydrilla verticillata
MTVARSVVAVNLSGSLQNRYPASSSVSCFLGKEYRCNSMLGFCGSGKLAFGANAPYSKIAATKPKPKLRPLKVNCMDFPRPDIDNTANFLEAAALSSSFRNSARPSKPLQVVIAGAGLAGLSTAKYLADAGHIPILLEARDVLGGKVAAWKDDDGDWYETGLHIFFGAYPNVQNLFGELGINDRLQWKEHSMIFAMPNKPGEFSRFDFPEVLPAPLNGIWAILKNNEMLTWPEKVQFAIGLLPAMIGGQPYVEAQDGLTVQEWMRKQGVPDRVNDEVFIAMSKALNFINPDELSMQCILIALNRFLQEKHGSKMAFLDGNPPERLCKPIADHIESLGGQVILNSRIQKIELNADKSVKHFVLTNGNIITGDAYVFATPVDILKLLLPEDWKEISYFKKLDKLVGVPVINVHIWFDRKLKNTYDHLLFSRSPLLSVYADMSVTCKEYYNPNQSMLELVFAPAEKWISCSDSEIINATMQELAKLFPDEISADQSKAKILKYHVVKTPRSVYKTVPDCEPCRPLQRSPIEGFYLAGDYTKQKYLASMEGAVLSGKLCAQAIVQDCSLLASRVQKSPQTLTIA

SEQ ID NO: 3 - DNA, 2293 bp, Glycine max
  CDS (221)..(1933)
gaattccttctacgtactgccgtggtgctttcaccactgcttaccactaaccttcctctctctctctgccgctgcaagcttggtactctcaactcaattctccaccttattcttttcacttcttcagctcttgttttttcccaaatctactttcaaagtgcctgaattctgcaacagtaatattaacactcctctcttttgttcaggctttatttccccaatggccgcttgtggctatatatctgctgccaacttcaattatctcgttggcgccagaaacatatccaaattcgcttcttcagacgccacaatttcgttttcatttggcgggagcgactcaatgggtcttactttgcgacccgctccgattcgtgctcctaagaggaaccatttctctcccttgcgtgtcgtttgcgtcgattatccacgcccagagctcgaaaacaccgttaatttcgttgaagctgcttacttgtcttccacctttcgtgcttctccgcgtcctctaaaacccttgaacatcgttattgccggtgcaggattggctggtttatcaactgcaaaatatttggctgatgctgggcataaacctatattgctggaagcaagagacgttctaggtggaaaggttgctgcatggaaagacaaggatggagactggtacgagacaggcctacacatcttttttggggcttacccttatgtgcagaacctttttggagaacttggcattaatgatcggttacaatggaaagagcattctatgatttttgctatgccaaataagcctggagagtttagtcgatttgattttcctgaagttcttccctccccattgaatggaatatgggcaatattgaggaacaatgagatgcttacatggccagagaaagtaaaatttgcaattgggcttctcccagctatgcttggcggacagccatatgttgaggctcaagatggtctttctgttcaagaatggatgaaaaagcagggcgtacctgaacgggtagctgatgaggtgttcatagcaatgtcaaaggcactaaacttcatcaatcctgatgaactttcaatgcaatgtatattgattgctttaaaccgatttcttcaggagaaacatggttctaagatggcctttttggatggcaatccacccgaaagactttgtatgccaatagttgattatattcagtccttgggtggtgaagttcatctaaattcgcgcattcaaaaaattgagctaaatgatgatggaacggtgaagagcttcttactaaataatgggaaagtgatggaaggggatgcttatgtgtttgcaactccagtggatattctgaagcttcttctaccagataactggaaagggattccatatttccagagattggataaattagttggcgtcccagtcataaatgttcacatatggtttgacagaaaactgaagaacacatatgatcaccttctctttagcagaagtccccttctgagtgtatatgctgacatgtcagtaacttgcaaggaatattatagcccaaaccagtcaatgttagagttggtttttgcaccagccgaagaatggatttcacgtagtgatgatgatattattcaagccacgatgactgagcttgccaaactctttcctgatgaaatttctgcagaccaaagcaaagcaaagattctcaagtaccatgttgttaaaacaccaaggtcggtttacaaaactgttccaaattgtgaaccttgtcgacccattcaaagatctcctatagaaggtttctatttagctggagattacacaaaacaaaaatatttagcttcaatggaaggcgctgttctttctgggaagctttgtgcacaggctattgtacaggattctgagctactagctactcggggccagaaaagaatggctaaagcaagtgttgtgtaacaaaaacaagaattgaaagagtcatggtagagtacaggagcatcatttcaactttggcattctttgtctgtggtcaggactcaggagaccttcaactttattagttcatacgaataaagaaaggctcagcttctgaaatttagctgcaccgtcgtcaactgtgtgcaataagctatacggaacaaacgacatgtgtcaactttaagtcagcccattgttttgttatcctccaattttctggatcaatgtttgtattggaaagaaatatgtcattattcaaacttgtttatatccactttttttatttatcaacatttgtcacaacctttcgttgagtaaaaaaaaaaaaaaaaagaattc

SEQ ID NO: 4 - protein, 570 aa, Glycine max
MAACGYISAANFNYLVGARNISKFASSDATISFSFGGSDSMGLTLRPAPIRAPKRNHFSPLRVVCVDYPRPELENTVNFVEAAYLSSTFRASPRPLKPLNIVIAGAGLAGLSTAKYLADAGHKPILLEARDVLGGKVAAWKDKDGDWYETGLHIFFGAYPYVQNLFGELGINDRLQWKEHSMIFAMPNKPGEFSRFDFPEVLPSPLNGIWAILRNNEMLTWPEKVKFAIGLLPAMLGGQPYVEAQDGLSVQEWMKKQGVPERVADEVFIAMSKALNFINPDELSMQCILIALNRFLQEKHGSKMAFLDGNPPERLCMPIVDYIQSLGGEVHLNSRIQKIELNDDGTVKSFLLNNGKVMEGDAYVFATPVDILKLLLPDNWKGIPYFQRLDKLVGVPVINVHIWFDRKLKNTYDHLLFSRSPLLSVYADMSVTCKEYYSPNQSMLELVFAPAEEWISRSDDDIIQATMTELAKLFPDEISADQSKAKILKYHVVKTPRSVYKTVPNCEPCRPIQRSPIEGFYLAGDYTKQKYLASMEGAVLSGKLCAQAIVQDSELLATRGQKRMAKASVV

SEQ ID NO: 5 - DNA, 2264 bp, Zea mays
  CDS (351)..(2066)
ctccaaatgcggaggtctcgactcttctctcttcctccatctttatcatcgccccacgtacacacccaattcctcgcaactgggctcccccgcctccacgacactgccccccgtctcaagtccgccgcctccattcttcagctctcctatcctccgcctagaatatcttcatcggtattttaccaacctggatcaatttactcacgatactctgaagcgtatacatatgccatatgggaaatgacttcatagctgtgggttgtcttatggctccttgaatttgcagtagtctgcctgtacctattggctgaagcagagctgacccccactttatcaagagttgctcaacgatggacactggctgcctgtcatctatgaatattactggagctagccagacaagatcttttgcggggcaacttcctcctcagagatgttttgcgagtagtcactatacaagctttgccgtgaaaaaacttgtctcaaggaataaaggaaggagatcacaccgtagacatcctgccttgcaggttgtctgcaaggattttccaagacctccactagaaagcacaataaactatttggaagctggacagctctcttcattttttagaaacagcgaacgccccagtaagccgttgcaggtcgtggttgctggtgcaggattggctggtctatcaacagcgaagtatctggcagatgctggccataaacccatattgcttgaggcaagagatgttttgggtggaaaggtagctgcttggaaggatgaagatggagattggtacgagactgggcttcatatattttttggagcttatcccaacatacagaatctgtttggcgagcttaggattgaggatcgtttgcagtggaaagaacactctatgatattcgccatgccaaacaagccaggagaattcagccggttcgatttcccagaaactttgccagcacctataaatgggatatgggccatattgagaaacaatgaaatgcttacttggccggagaaggtgaagtttgcaatcggacttctgccagcaatggttggtggtcaaccttatgttgaagctcaagatggcttaaccgtttcagaatggatgaaaaagcagggtgttcctgatcgggtgaacgatgaggtttttattgcaatgtccaaggcactcaatttcataaatcctgatgagctatctatgcagtgcattttgattgctttgaaccgatttcttcaggagaagcatggttctaaaatggcattcttggatggtaatccgcctgaaaggctatgcatgcctattgttgatcacattcggtctaggggtggagaggtccgcctgaattctcgtattaaaaagatagagctgaatcctgatggaactgtaaaacacttcgcacttagtgatggaactcaaataactggagatgcttatgtttgtgcaacaccagtcgatatcttcaagcttcttgtacctcaagagtggagtgaaattacttatttcaagaaactggagaagttggtgggagttcctgttatcaatgttcatatatggtttgacagaaaactgaacaacacatatgaccaccttcttttcagcaggagttcacttttaagtgtctatgcagacatgtcagtaacctgcaaggaatactatgacccaaaccgttcaatgctggagttggtctttgctcctgcagacgaatggattggtcgaagtgacactgaaatcatcgatgcaactatggaagagctagccaagttatttcctgatgaaattgctgctgatcagagtaaagcaaagattcttaagtatcatattgtgaagacaccgagatcggtttacaaaactgtcccaaactgtgagccttgccggcctctccaaaggtcacctatcgaaggtttctatctagctggtgattacacaaagcagaaatacctggcttctatggaaggtgcagtcctatccgggaagctttgtgcccagtccatagtgcaggattatagcaggctcgcactcaggagccagaaaagcctacaatcaggagaagttcccgtcccatcttagttgtagttggctttagctatcgtcatccccactgggtgctatcttatctcctatttcaatgggaacccacccaatggtcatgttggagacaacacctgttatggtcctttgaccatctcgtggtgactgtagttgatgtcatattcggatatatatgtaaaaggacctgcatagcaattgttagaccttggaaaaaaa

SEQ ID NO: 6 - protein, 571 aa, Zea mays
MDTGCLSSMNITGASQTRSFAGQLPPQRCFASSHYTSFAVKKLVSRNKGRRSHRRHPALQVVCKDFPRPPLESTINYLEAGQLSSFFRNSERPSKPLQVVVAGAGLAGLSTAKYLADAGHKPILLEARDVLGGKVAAWKDEDGDWYETGLHIFFGAYPNIQNLFGELRIEDRLQWKEHSMIFAMPNKPGEFSRFDFPETLPAPINGIWAILRNNEMLTWPEKVKFAIGLLPAMVGGQPYVEAQDGLTVSEWMKKQGVPDRVNDEVFIAMSKALNFINPDELSMQCILIALNRFLQEKHGSKMAFLDGNPPERLCMPIVDHIRSRGGEVRLNSRIKKIELNPDGTVKHFALSDGTQITGDAYVCATPVDIFKLLVPQEWSEITYFKKLEKLVGVPVINVHIWFDRKLNNTYDHLLFSRSSLLSVYADMSVTCKEYYDPNRSMLELVFAPADEWIGRSDTEIIDATMEELAKLFPDEIAADQSKAKILKYHIVKTPRSVYKTVPNCEPCRPLQRSPIEGFYLAGDYTKQKYLASMEGAVLSGKLCAQSIVQDYSRLALRSQKSLQSGEVPVPS

SEQ ID NO: 7 - DNA, 2027 bp, Oryza sativa
  CDS (5)..(1705)
gtttatgacagcatctgccagatattttgcaggacaacttcctactcataggtgcttcgcaagtagcagcatccaagcactgaaaggtagtcagcatgtgagctttggagtgaaatctcttgtcttaaggaataaaggaaaaagattccgtcggaggctcggtgctctacaggttgtttgccaggactttccaagacctccactagaaaacacaataaactttttggaagctggacaactatcctcatttttcagaaacagtgaacaacccactaaaccattacaggtcgtgattgctggagcaggattagctggtttatcaacggcaaaatatctggcagatgctggtcataaacccatattgcttgaggcaagggatgttttgggtggaaagatagctgcttggaaggatgaagatggagattggtatgaaactgggcttcatatcttttttggagcttatcccaacatacagaacttgtttggcgagcttggtattaatgatcggttgcaatggaaggaacactccatgatatttgccatgccaaacaagccaggagaatccagccggtttgattttcctgaaacattgcctgcacccttaaatggaatatgggccatactaagaaacaatgaaatgctaacttggccagagaaggtgaagtttgctcttggacttttgccagcaatggttggtggccaagcttatgttgaagctcaagatggttttactgtttctgagtggatgaaaaagcagggtgttcctgatcgagtgaacgatgaagttttcattgcaatgtcaaaggcacttaatttcataaatcctgatgagttatccatgcagtgcattctgattgctttaaaccgatttcttcaggagaagcatggttctaagatggcattcttggatggtaatcctcctgaaaggttatgcatgcctattgttgaccatgttcgctctttgggtggtgaggttcggctgaattctcgtattcagaaaatagaacttaatcctgatggaacagtgaaacactttgcacttaccgatggaactcaaataactggagatgcttatgtttttgcaacaccagttgatatcttgaagcttcttgtacctcaagagtggaaagaaatatcttatttcaagaagctggagaagttggtgggagttcctgttataaatgttcatatatggtttgatagaaaactgaagaacacatatgaccaccttcttttcagcaggagttcacttttaagtgtttatgcggacatgtcagtaacttgcaaggaatactatgatccaagccgttcaatgctggagttggtctttgctcctgcagaggaatgggttggacggagtgacactgaaatcatcgaagcaactatgcaagagctagccaagctatttcctgatgaaattgctgctgatcagagtaaagcaaagattctgaagtatcatgttgtgaagacaccaagatctgtttacaagactatcccggactgtgaaccttgccgacctctgcaaagatcaccgattgaagggttctatctagctggtgactacacaaagcagaaatatttggcttcgatggagggtgcagttctatctgggaagctttgtgctcagtctgtagtggaggattataaaatgctatctcgtaggagcctgaaaagtctgcagtccgaagttcctgttgcctcctagttgtagtcaggactattcccaatggtgtgtgtgtcatcatcccctagtcagtttttttctatttagtgggtgcccaactctccaccaatttacacatgatggaacttgaaagatgcctattttggtcttatcatatttctgtaaagttgatttgtgactgagagctgatgccgatatgccacgctggagaaaaagaacattatgtaaaacgacctgcatagtaattcttagacttttgcaaaaggcaaaaggggtaaagcgaccttttttttctatgtgaagggattaagagaccttaaaaaaaaaaaaaaaaaaaaaaaaa

SEQ ID NO: 8 - protein, 566 aa, Oryza sativa
MTASARYFAGQLPTHRCFASSSIQALKGSQHVSFGVKSLVLRNKGKRFRRRLGALQVVCQDFPRPPLENTINFLEAGQLSSFFRNSEQPTKPLQVVIAGAGLAGLSTAKYLADAGHKPILLEARDVLGGKIAAWKDEDGDWYETGLHIFFGAYPNIQNLFGELGINDRLQWKEHSMIFAMPNKPGESSRFDFPETLPAPLNGIWAILRNNEMLTWPEKVKFALGLLPAMVGGQAYVEAQDGFTVSEWMKKQGVPDRVNDEVFIAMSKALNFINPDELSMQCILIALNRFLQEKHGSKMAFLDGNPPERLCMPIVDHVRSLGGEVRLNSRIQKIELNPDGTVKHFALTDGTQITGDAYVFATPVDILKLLVPQEWKEISYFKKLEKLVGVPVINVHIWFDRKLKNTYDHLLFSRSSLLSVYADMSVTCKEYYDPSRSMLELVFAPAEEWVGRSDTEIIEATMQELAKLFPDEIAADQSKAKILKYHVVKTPRSVYKTIPDCEPCRPLQRSPIEGFYLAGDYTKQKYLASMEGAVLSGKLCAQSVVEDYKMLSRRSLKSLQSEVPVAS

(19) United States
(12) Patent Application Publication
(10) Pub. No.: US 2011/0098180 A1
(43) Pub. Date: Apr. 28, 2011

(54) Herbicide-Resistant Plants, And Polynucleotides And Methods For Providing Same

(75) Inventors: Albrecht Michel, Loerrach (DE); Michael D. Netherland, Gainesville, FL (US); Brian E. Scheffler, Stoneville, MS (US); Franck E. Dayan, Oxford, MS (US); Renee S. Arias De Ares, Leland, MS (US)

(73) Assignee: United States Department of Agriculture, Washington, DC (US)

(21) Appl. No.: 10/521,478

(22) PCT Filed: Jul. 17, 2003
(86) PCT No.: PCT/US2003/022295

§ 371 Date: Sep. 17, 2007

Related U.S. Application Data
(60) Provisional application No. 60/396,539, filed on Jul. 17, 2002.
(60) Provisional application No. 60/401,579, filed on Aug. 7, 2002.

(51) Int. Cl. A01N 25/00; C07H 21/04; C12N 15/63; C12N 9/02; A01H 5/00; C12N 15/82; A01H 1/00; C12Q 1/68; A01P 13/00

(52) U.S. Cl. 504/116.1; 536/23.2; 435/320.1; 435/189; 800/300; 800/300.1; 800/278; 435/6

(57) ABSTRACT

Described are polynucleotides having nucleotide sequences encoding mutant plant phytoene desaturase proteins that are resistant the bleaching herbicides that act on phytoene desaturase, and related nucleic acid constructs, plants and methods.

### REFERENCE TO RELATED APPLICATIONS

This application claims the benefit of U.S. Patent Applications Ser. Nos. 60/396,539 and 60/401,579 filed Jul. 17, 2002 and Aug. 7, 2002, respectively, each of which is incorporated herein by reference in its entirety.

### BACKGROUND OF THE INVENTION

The present invention relates generally to modified plant proteins and polynucleotides encoding them. More particularly the present invention relates to modified plant phytoene desaturase genes and proteins, and their use to generate herbicide resistant plants.

The photosynthetic membranes of plants contain carotenoids. Carotenoids protect chlorophyll against photooxidative damage by singlet oxygen, and also act as accessory pigments in photosynthetic light harvesting. The first committed step in carotenoid biosynthesis is the condensation of two molecules of geranylgeranyl pyrophosphate (GGPP) to yield phytoene.

Desaturation of phytoene, to insert four double bonds, forms lycopene, and further cyclization reactions lead to the generation of Beta-carotene. Phytoene desaturase (PDS) is an enzyme that mediates the first two steps of desaturation of phytoene. A number of commercial herbicides directed at inhibiting this enzyme have been developed, e.g. norflurazon, fluridone, and fluorochloridone. This inhibition results in an observable bleaching symptom, and thus these herbicides have been termed “bleaching herbicides”.

The literature contains few reports of organisms resistant to bleaching herbicides. Hirschberg et al, 1996, WO9628014, describes a gene from an Erwinia species transformed into cyanobacteria, specifically Synechococcus PCC 7942 and Synechocystis PCC 6803. These were used to provide screens for beta-carotene biosynthesis and for mutants resistant to bleaching herbicides of the trialkylamine family.

Screening for bleaching activity is described by Sandmann, G., Schmidt A., Linden, H., Boger, P., Weed Science, 39, pp. 474-479 (1991) as a means to discover new herbicides with different core structures which inhibit PDS. Windhoevel et al, 1994 describe a screen involving genes coding for PDS of Erwinia uredovora introduced into the cyanobacterium Synechococcus as a convenient experimental model for discovering resistance to herbicides (see, Windhoevel, U. Geiges, B. Sandman, G. Boeger, P., Pestic. Biochem. Physiol., 1994, 49,1, p. 63-71; Windhoevel, U., Geiges, B. Sandman, G. Boeger, P., Plant Physiol., 1994, 104,1, p. 6371). The functionality of the heterologously expressed PDS in the transformants was demonstrated in assays. Other references such as Babczinski, P., Sandmann, G., Schmidt, R., Shiokawa, Kozo, Yasui, Katzucsmi, Pestic. Biochem. Physiol., 1995, 52,1, p 33-44, identify a new herbicide class inhibiting PDS based on a screen utilizing the unicellular cyanobacteria Anacystis. Chamowitz, D. Sandmann, G. Hirschberg, J., J. Biol. Chem., 1993, 268,23, p. 17348-53, describes a cell-free carotegenic assay to identify herbicide resistant algal PDS mutants. Inhibition of carotenoid biosynthesis by herbicidal phenoxybenzamide derivatives was investigated in a cell-free in vitro assay using the cyanobacteria Aphanocapsa by Clarke, I. E. Sandmann, G. Brawley, P. M. Boeger, P., Pestic. Biochem. Physiol., 1985, 23,3, p. 335-340, and subsequently by Kowalczyl-Schroder, S. Sandmann, G., Pestic. Biochem. Physiol., 1992, 42,1, p. 7-12. Sandmann, G., Schmidt A., Linden, H., Boger, P., Weed Science, 39, pp. 474-479 (1991), describes a non-radioactive cell-free assay to quantitatively determine inhibition of plant PDS by bleaching herbicides. They further developed a cyanobacterial PDS assay system, a mode of action assay utilizing the cyanobacteria Anacystis, and assays using algal cells.

Linden, H., Sandmann, G., Chamovitz, D., Hirschberg, J., Booger, P., Pesticide Biochemistry and Physiology, 36, pp. 46-51 (1990), reported cyanobacteria Synechococcus PCC 7942 mutants selected against the bleaching herbicide norflurazon. One strain exhibited cross-resistance against another bleaching herbicide fluorochloridone, but the other three strains did not show cross-resistance against other PDS inhibitors. Sandmann, G., Schmidt A., Linden, H., Boger, P., Weed Science, 39, pp. 474-479 (1991), reported on mutants from Synechococcus PCC 7942, which were selected for tolerance to various bleaching herbicides. A mutant NFZ4 established a high degree of cross-resistance to both norflurazon and fluorochloridone, but not to fluridone. Chamowitz, D. Sandmann, G. Hirschberg, J., J. Biol. Chem., 1993, 268,23, p. 17348-53, cloned and sequenced a PDS gene from the cyanobacteria Synechococcus PCC 7942, also resistant to the bleaching herbicide norflurazon. The identified mutant is a Val=>Gly change at position 403 in the Synechococcus but not Synechocystis PDS protein. Breitenbach, J.; Fernandez Gonzalez, B.; Vioque, A.; Sandmann, G. A higher-plant type z-carotene desaturase in the cyanobacterium Synechocystis PCC6803. Plant Molecular Biology 1998, 36, 725-732, reported bacterial and fungal PDS as a target for bleaching herbicides, and discussed the identification of cyanobacterial mutants with resistance to specific compounds and their cross-resistance to other bleaching herbicides.

A spontaneous cyanobacteria Synechocystis mutant, strain AV4, which is resistant to norflurazon, was isolated from cyanobacterium Synechocystis PC 6803. DNA isolated from the mutant AV4 can transform wild-type cells to norflurazon resistance with high frequency. Martinez-Ferez, I.; Vioque, A.; Sandmann, G. Mutagenesis of an amino acid responsible in phytoene desaturase from Synechocystis for binding of the bleaching herbicide norflurazon. Pesticide Biochemistry and Physiology 1994, 48, 185-190), identified three distinct Synechocystis mutants selected against norflurazon, and showed modification of the same amino acid of PDS into three different ones. In all cases, the same amino acid Arg¹⁹⁵ was modified either into Cys, Pro or Ser. The degree of resistance was highest when Arg was changed into Ser.

In light of this background, there remain needs for new modified PDS polynucleotides and proteins, especially from higher plants, that may be used, inter alia, to provide bleaching herbicide-resistant plants, selection markers, and methods for selectively controlling weeds in cultivated areas. The present invention is addressed to these needs.

### SUMMARY OF THE INVENTION

Mutant plant phytoene desaturase genes have been discovered that confer resistance to bleaching herbicides that act upon plant phytoene desaturase enzymes. The identification of such novel phytoene desaturase mutants in higher plants enables the. generation of a wide variety of herbicide-resistant plants. Such plants can be generated, for example, by the introduction of a polynucleotide encoding a mutant plant phytoene desaturase enzyme or by mutation of the native phytoene desaturase gene of a plant. In preferred embodiments, the mutant phytoene desaturase enzymes exhibit unexpected cross-resistance patterns to a number of bleaching herbicidal compounds.

Accordingly, one embodiment of the present invention provides an isolated polynucleotide having a nucleotide sequence encoding a mutant plant phytoene desaturase enzyme with increased resistance to one or more bleaching herbicides. Preferred polynucleotides of the invention will encode a plant phytoene desaturase enzyme having at least one point mutation relative to the corresponding wild-type enzyme, providing the increased bleaching herbicide resistance. More preferred polynucleotides will be selected from:

(a) polynucleotides encoding a plant phytoene desaturase enzyme having an amino acid sequence at least 80% identical to amino acids 109 to 580 of SEQ ID NO: 2 (the wild-type phytoene desaturase sequence from hydrilla), said amino acid sequence having a point mutation corresponding to one or more of positions 304, 425, 509, and 542 of SEQ ID NO: 2.

(b) polynucleotides encoding a plant phytoene desaturase enzyme having an amino acid sequence at least 80% identical to amino acids 97 to 570 of SEQ ID NO: 4 (the.wild-type sequence from soybean), said amino acid sequence having a point mutation corresponding to one or more of positions 294, 415, 499, and 532 of SEQ ID NO: 4;

(c) polynucleotides encoding a plant phytoene desaturase enzyme having an amino acid sequence at least 80% identical to amino acids 97 to 571 of SEQ ID NO: 6 (the wild-type sequence from maize), said amino acid sequence having a point mutation corresponding to one or more of positions 292, 413, 497 and 530 of SEQ ID NO: 6; and

(d) polynucleotides encoding a plant phytoene desaturase enzyme having an amino acid sequence at least 80% identical to amino acids 93 to 566 of SEQ ID NO: 8 (the wild-type sequence from rice), said amino acid sequence having a point mutation corresponding to one or more of positions 288, 409, 493, and 526 of SEQ ID NO: 8; and

(e) polynucleotides encoding a mutant plant phytoene desaturase enzyme with increased resistance to one or more bleaching herbicides, wherein the polynucleotides have a nucleotide sequence at least about 60% identical to nucleotides 324 to 1748 of SEQ ID NO: 1, nucleotides 509 to 1933 of SEQ ID NO: 3, nucleotides 633 to 2066 of SEQ ID NO: 5, or nucleotides 275 to 1705 of SEQ ID NO: 7; preferably, these polynucleotides encode mutant phytoene desaturase enzymes having one or more amino acid point mutations as discussed above.

Another embodiment of the present invention provides a nucleic acid construct including polynucleotide as described above. The construct is preferably a vector including the polynucleotide operably coupled to a regulatory sequence such as a promoter.

Another embodiment of the invention provides a purified, mutant plant PDS enzyme exhibiting increased resistance to one or more bleaching herbicides. Preferred enzymes will have an amino acid sequence at least about 80% identical to any one of SEQ ID NOs: 2, 4, 6, and 8 and will contain at least one amino acid point mutation providing the increased resistance, for example one or more of the specific point mutation described above.

Another embodiment of the invention provides an herbicide-resistant crop plant including in its genome an expressible polynucleotide encoding a mutant plant PDS enzyme conferring resistance to one or more bleaching herbicides. Desirably, the polynucleotide in such plants encodes a mutant PDS enzyme that is at least 80% identical to any one of SEQ ID NOs: 2, 4, 6, and 8, and/or the PDS polynucleotide is at least about 60% identical to any one of SEQ ID NOs: 1, 3, 5, and 7. The invention is applied with preference to major monocot and dicot crops such as maize, soybean, rice, wheat, barley, cotton and canola.

The invention also provides a method for making an herbicide-resistant plant, comprising modifying a plant to incorporate in its genome a sequence of nucleotides encoding a modified plant phytoene desaturase enzyme having increased resistance to one or more bleaching herbicides, the modified plant phytoene desaturase enzyme having at least one amino acid point mutation that provides said increased resistance. In certain forms, methods of the invention may include the steps of transforming plant material with a polynucleotide or nucleic acid construct of the invention; selecting the thus transformed material; and regenerating the thus selected material into a morphologically normal fertile whole plant.

The invention still further provides a method of selectively controlling weeds in a cultivated area, the area comprising weeds and plants of the invention or the herbicide-resistant progeny thereof, the method comprising applying to the field a bleaching herbicide in an amount sufficient to control the weeds without substantially affecting the plants.

The novel mutant plant phytoene desaturase polynucleotides of the invention may also be used as selectable markers for other polynucleotides to be incorporated such as herbicide, fungal and insect resistance genes as well as output trait genes, wherein the appropriate bleaching herbicide is used to provide the selection pressure. Such a selectable marker system for nuclear or plastidic transformation can be used for major monocot and dicot crops identified above, as well as other plants or tissues.

The invention also provides access to screening methods, including high throughput screening methods, for candidate herbicidal compounds, using mutant PDS enzymes and cells, tissues or plants expressing them.

Additional preferred embodiments as well as features and advantages of the invention will be apparent from the descriptions herein.

### BRIEF DESCRIPTION OF THE SEQUENCE LISTING

SEQ ID NOs: 1 and 2 show the nucleotide sequence and deduced amino acid sequence for a wild-type phytoene desaturase precursor from Hydrilla verticillata. The putative mature protein spans from amino acids 109 to 580; the putative transit peptide spans from amino acids 1 to 108.

SEQ ID NOs: 3 and 4 show the nucleotide sequence and deduced amino acid sequence for a wild-type phytoene desaturase precursor from Glycine max (soybean). The putative mature protein spans from amino acids 97 to 570; the putative transit peptide spans from amino acids 1 to 96.

SEQ ID NOs: 5 and 6 show the nucleotide sequence and deduced amino acid sequence for a wild-type phytoene desaturase precursor from Zea mays (maize). The putative mature protein spans from amino acids 97 to 571; the putative transit peptide spans from amino acids 1 to 96.

SEQ ID NOs: 7 and 8 show the nucleotide sequence and deduced amino acid sequence for a wild-type phytoene desaturase precursor from Oryza sativa (rice). The putative mature protein spans from amino acids 93 to 566; the putative transit peptide spans from amino acids 1 to 92.

### DESCRIPTION OF THE PREFERRED EMBODIMENTS

For the purpose of promoting an understanding of the principles of the invention, reference will now be made to certain preferred embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

As disclosed above, the present invention provides novel polynucleotides encoding mutant, bleaching herbicide-resistant plant PDS enzymes and novel uses thereof, bleaching herbicide-resistant plant PDS enzymes, bleaching herbicide-resistant plants, and selection and screening methods.

As used herein, the term “polynucleotide” refers to a linear segment of single- or double-stranded deoxyribonucleic acid (DNA) or ribonucleic acid (RNA), which can be derived from any source. Preferably, the polynucleotide of the present invention is a segment of DNA.

The term “plant” refers to a photosynthetic organism including algae, mosses, ferns, gymnosperms, and angiosperms. The term, however, excludes, prokaryotic and eukaryotic microorganisms such as bacteria, yeast, and fungi.

“Plant cell” includes any cell derived from a plant, including undifferentiated tissue such as callus or gall tumor, as well as protoplasts, and embryonic and gametic cells.

The term “nucleotide sequence” refers to a polymer of DNA or RNA which can be single- or double-stranded, optionally containing synthetic, non-natural, or altered nucleotides capable of incorporation into DNA or RNA polymers.

The term “nucleic acid construct” refers to a plasmid, virus, autonomously replicating sequence, phage or linear segment of a single- or double-stranded DNA or RNA, derived from any source, which is capable of introducing a polynucleotide into a biological cell.

“Regulatory nucleotide sequence”, as used herein, refers to a nucleotide sequence located 5′ and/or 3′ to a nucleotide sequence whose transcription and expression is controlled by the regulatory nucleotide sequence in conjunction with the protein synthetic apparatus of the cell. A “regulatory nucleotide sequence” can include a promoter region, as that term is conventionally employed by those skilled in the art. A promoter region can include an association region recognized by an RNA polymerase, one or more regions which control the effectiveness of transcription initiation in response to physiological conditions, and a transcription initiation sequence.

“Transit peptide” refers to a signal polypeptide which is translated in conjunction with a polypeptide, forming a polypeptide precursor. In the process of transport to a selected site within the cell, for example, a chloroplast, the transit peptide can be cleaved from the remainder of the polypeptide precursor to provide an active or mature protein.

“Bleaching herbicide,” as used herein, refers to a herbicidal compound that inhibits phytoene desaturase in plant cells or whole plants.

“Resistance” refers to a capability of an organism or cell to grow in the presence of selective concentrations of an inhibitor.

In relation to particular enzymes or proteins, “sensitive” indicates that the enzyme or protein is susceptible to inhibition by a particular inhibiting compound at a selective concentration, for example, a herbicide.

In relation to particular enzymes or proteins, “resistant” indicates that the enzyme or protein, as a result of a different protein structure, expresses activity in the presence of a selective concentration of a specific inhibitor, which inactivates sensitive variants of the enzyme or protein.

Nucleotides are indicated herein by their bases by the following standard abbreviations:

A=adenine;

C=cytosine;

T=thymine;

G=guanine.

Amino acid residues are indicated at some points herein by the following standard abbreviations:

Ala=alanine;

Cys=cysteine;

Asp=aspartic acid;

Glu=glutamic acid;

Phe=phenylalanine;

Gly=glycine;

His=histidine;

Ile=isoleucine;

Lys=lysine;

Leu=leucine;

Met=methionine;

Asn=asparagine;

Pro=proline;

Glu=glutamine;

Arg=arginine;

Ser=serine;

Thr=threonine;

Val=valine;

Trp=tryptophan; and

Tyr=tyrosine.

The term “amino acids” as used herein is meant to denote the above-recited natural amino acids and functional equivalents thereof.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions.times.100).

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to AIP-6 nucleic acid molecules of the invention. BLAST protein searches can be performed with the XBLAST program, score=50, wordlength=3 to obtain amino acid sequences homologous to AIP-6 protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

Mutant plant phytoene desaturase (PDS) genes have been discovered that confer increased resistance to bleaching herbicides. Plant PDS genes and their encoded plant PDS proteins exhibit extremely high identity among higher plants, including major monocot and dicot crop plants such as maize, rice, and soybean. Accordingly, similar mutations in highly identical plant PDS genes/proteins are expected to confer similar resistance to bleaching herbicides.

In work to date, several biotypes of the aquatic plant Hydrilla verticillata (hydrilla) that had evolved resistance to the PDS-inhibiting herbicide, Fluridone, were identified and characterized. The PDS genes from these resistant plants have been cloned and sequenced, and provide novel eukaryotic polynucleotides encoding for eurkaryotic plant PDS enzymes that confer resistance to PDS-inhibiting herbicides. Sequence analysis demonstrated that the wild-type (herbicide-sensitive) PDS precursor enzyme from hydrilla (SEQ ID NO: 2) has an arginine residue at position 304. This arginine residue has been converted to histidine cysteine or serine in the genes from the resistant biotypes. In particular, the wild-type codon for position 304 of the hydrilla PDS precursor gene (SEQ ID NO: 1) is CGT, which encodes for arginine. In the resistant biotypes there are various single nucleotide mutations that result in single amino acid point mutations at position 304 (CAT→histidine, TGT→cysteine, and AGT→serine) of the PDS precursor protein. These mutations rendered the PDS enzyme resistant to normal rates of fluridone.

To demonstrate that these mutations originally discovered in hydrilla impart resistance in crop plants having highly similar PDS genes, the ³⁰⁴Arg→His substitution found in hydrilla was introduced in the corresponding position (codon and amino acid 292) in the maize PDS precursor sequence (see SEQ ID NOs: 5 and 6). This modified maize PDS enzyme was substantially more resistant to the bleaching herbicide fluridone than the wild-type maize PDS enzyme, confirming that corresponding mutations in highly similar plant PDS enzymes provide similar herbicide resistance.

To test whether other mutations lead to resistance to fluridone (and potential cross-resistance to other inhibitors of phytoene desaturase), other amino acid substitutions were made at position 304 of the hydrilla PDS precursor enzyme. With single nucleotide changes, the codon at position 304 (CGT) can be changed to the following amino acids: first position nucleotide mutation: cysteine (TGT), serine (AGT), glycine (GGT); second position nucleotide mutation; leucine (CTT), proline (CCT), histidine (CAT); any changes in the third position would result in the same arginine residue. Three of these substitutions (histidine, cysteine, serine) existed in the identified resistant forms of hydrilla. Thus, glycine, proline, and leucine substitutions for arginine were introduced at this position as representative of additional single nucleotide substitutions. Further resistant biotypes may result from multiple nucleotide substitutions at this codon position of the hydrilla PDS gene or corresponding positions in PDS genes of other plants. Thus, additional amino acids were introduced at position 304 as follows:

| Amino Acid | Codon |
|---|---|
| Alanine | GCT |
| Valine | GTT |
| Isoleucine | ATT |
| Methionine | ATG |
| Phenyalanine | TTC |
| Tryptophan | TGG |
| Threonine | ACT |
| Asparagine | AAT |
| Glutamine | CAG |
| Tyrosine | TAT |
| Lysine | AAG |
| Aspartic Acid | GAT |
| Glutamic Acid | GAG |

The resulting PDS enzymes were all evaluated for resistance to fluridone by in vitro enzyme inhibition analysis. The results are shown in Table 1.

TABLE 1
Fluridone Enzyme Inhibition Assays Hydrilla
PDS Arg³⁰⁴ → Substitute Amino Acid

| Substitute Amino Acid | I₅₀ (nM) | R/S |
|---|---|---|
| Glycine | 640 | 3.2 |
| Alanine | 4,500 | 22.5 |
| Valine | 2,200 | 11 |
| Leucine | 3,200 | 16 |
| Isoleucine | 2,000 | 10 |
| Methionine | 2,300 | 11.5 |
| Proline | * | * |
| Phenyalanine | 530 | 2.6 |
| Tryptophan | * | * |
| Threonine | 10,000 | 40 |
| Asparagine | 630 | 3.2 |
| Glutamine | 3,800 | 19 |
| Tyrosine | 810 | 4 |
| Lysine | 1,000 | 5 |
| Arginine | 200 | 1 |
| Aspartic acid | 2,000 | 10 |
| Glutamic acid | 310 | 1.5 |

* Not Active in Work to Date

The expression constructs in Table 1 were derived from an original clone of the susceptible PDS gene from Hydrilla and then the Arg³⁰⁴ was mutated to the listed amino acid. Expression was under the control of the lac promoter but not in frame with the initiation codon of the LacZalpha-ccdB gene in the pCR4-TOPO vector (Invitrogen Inc., CA, Cat. #K4575-01).

As can be seen, with the exception of proline and tryptophan, all amino acid substitutions tested at position 304 increased the resistance of the PDS enzyme to fluridone.

To explore whether mutations at this position of the hydrilla PDS enzyme and corresponding positions of other plant PDS enzymes confer cross resistance to multiple bleaching herbicides, hydrilla PDS enzymes with position 304 arginine histidine, cysteine, serine or threonine mutations were evaluated in vitro for resistance to Beflubutamid, Diflufenican, Fluorochloridone, Fluridone, Flurtamone, Norflurazon and Picolinafen. The results are set forth in Table 2.

TABLE 2
Cross Resistance to Bleaching Herbicides

| | Arginine | | Cysteine | | Histidine | | Serine | | Threonine | |
|---|---|---|---|---|---|---|---|---|---|---|
| Compounds | I₅₀ | R/S | I₅₀ | R/S | I₅₀ | R/S | I₅₀ | R/S | I₅₀ | R/S |
| Beflubutamid | 4.3 | 1 | 3.5 | 0.8 | 3.0 | 0.7 | 2.6 | 0.6 | 1.2 | 0.3 |
| Diflufenican | 5.8 | 1 | 2.1 | 0.4 | 2.0 | 0.4 | 1.1 | 0.2 | 1.0 | 0.2 |
| Fluorochloridone | 12.2 | 1 | 44.3 | 3.6 | 14.0 | 1.1 | 23.0 | 1.9 | 52.0 | 4.3 |
| Fluridone | 1.8 | 1 | 5.7 | 3.2 | 5.0 | 2.8 | 9.9 | 5.5 | 18.1 | 10.0 |
| Flurtamone | 2.8 | 1 | 5.3 | 1.9 | 3.3 | 1.2 | 5.9 | 2.1 | 8.1 | 2.9 |
| Norflurazon | 3.1 | 1 | 89.4 | 28.8 | 5.0 | 1.6 | 54.9 | 17.7 | 161.2 | 52.0 |
| Piconilafen | 5.6 | 1 | 2.4 | 0.4 | 3.0 | 0.5 | 2.3 | 0.4 | 2.7 | 0.5 |

I₅₀ is expressed as μM

The expression constructs in Table 2 were derived from an original clone of the susceptible PDS gene from Hydrilla and then the Arg³⁰⁴ was mutated to the listed amino acid. Expression was under the control of the lac promoter but in frame with the initiation codon of the LacZalpha-ccdB gene in the pCR4-TOPO vector (Invitrogen Inc., CA, Cat. #K4575-01). As the results show, the single point mutations provided plant PDS enzymes having cross-resistance to multiple PDS inhibiting herbicides.

In further cross-resistance testing similar to that described above, an alternative production and purification protocol was used in the preparation of the mutant PDS proteins, and the activity of the proteins was again tested using the same testing protocol. The alternative approach utilized His-tagging and column purification of the mutant PDS protins. The results are shown in. Table 2A below.

TABLE 2A

| | Arginine | | Cysteine | | Histidine | | Serine | | Threonine | |
|---|---|---|---|---|---|---|---|---|---|---|
| Compounds | I₅₀ | R/S | I₅₀ | R/S | I₅₀ | R/S | I₅₀ | R/S | I₅₀ | R/S |
| Beflubutamid | 72 | 1 | 232 | 3.2 | 126 | 1.75 | 465 | 6.5 | 106 | 1.5 |
| Diflufenican | 101 | 1 | 241 | 2.4 | 103 | 1.0 | 223 | 2.2 | 104 | 1.0 |
| Fluorochloridone | 130 | 1 | 283 | 2.2 | 364 | 2.8 | 518 | 4.0 | 210 | 1.6 |
| Fluridone | 305 | 1 | 799 | 2.6 | 1470 | 4.8 | 611 | 2.0 | 2373 | 7.8 |
| Flurtamone | 300 | 1 | 1148 | 3.8 | 630 | 2.1 | 707 | 2.4 | 1784 | 5.9 |
| Norflurazon | 50 | 1 | 1872 | 37.0 | 486 | 9.7 | 4104 | 82.0 | 1509 | 30.0 |
| Piconilafen | 327 | 1 | 96 | 0.3 | 112 | 0.3 | 62 | 0.2 | 60 | 0.2 |

I₅₀ is expressed as μM.

The results presented in Table 2A confirm that the mutations at Arg³⁰⁴ altered the activity of the PDS proteins in the presence of the bleaching herbicides. The general character of the resistance to the bleaching herbicides for the various mutations was similar to that in Table 2, with the exception of that shown for Beflubutamid and Diflufenican, which proved to exhibit increased resistance in the work presented in Table 2A.

To explore whether mutations at positions other than 304 of the hydrilla PDS enzyme and corresponding positions of other plant PDS enzymes also confer herbicide resistance, mutations discovered in Synechococcus strain PCC7942 (Leu³²⁰→Pro; Val⁴⁰³→Gly; Leu⁴³⁶→Arg) were introduced into the hydrilla PDS sequence at the corresponding locations, and the resulting PDS enzymes tested for resistance. The results are set forth in Table 3.

TABLE 3
Fluridone Enzyme Inhibition Assays Other Amino
Acid Substitutions in Hydrilla PDS

| Substitution | I₅₀ | R/S |
|---|---|---|
| Leu⁴²⁵ → Pro | 320 | 1.6 |
| Val⁵⁰⁹ → Gly | 2,900 | 14.5 |
| Leu⁵⁴² → Arg | 900 | 4.5 |

As the results demonstrate, these mutations also provided plant PDS enzymes having increased resistance to fluridone. This demonstrated resistance pattern is unexpected and evidences differences in the activities of Synechocystis PDS enzymes and the plant PDS enzymes. For example, in prior work with Synechocystis PDS enzymes, a mutation corresponding to the Leu⁵⁴²→Arg mutation had provided a high level of resistance to norflurazon but had failed to provide resistance to fluridone.

The present invention provides isolated polynucleotides encoding plant PDS enzymes that have increased resistance to one or more bleaching herbicides. Preferred polynucleotides of the invention will have a nucleotide sequence encoding a PDS enzyme having an amino acid sequence with at least 80% identity to amino acids 109 to 580 of SEQ ID NO: 2, to amino acids 97 to 570 of SEQ ID NO: 4, to amino acids 97 to 571 of SEQ ID NO: 6, or to amino acids 93 to 566 of SEQ ID NO: 8. More preferably, polynucleotides of the invention will encode a mutant PDS enzyme having at least about 90% identity to any one of the designated amino acid ranges of said sequences, and most preferably at least about 95% identity to any one of the designated amino acid ranges of said sequences. Polynucleotides of the invention will encode these PDS enzymes having at least one amino acid change relative to the corresponding wild-type plant PDS enzyme, especially having at least one of the following characteristics:

a) The polynucleotide encodes an amino acid other than arginine at position 304 of SEQ ID NO: 2; at position 294 of SEQ ID NO: 4; at position 292 of SEQ ID NO: 6; or at position 288 of SEQ ID NO: 8. The amino acid can be glycine, alanine, valine, leucine, isoleucine, methionine, phenyalanine, serine, threonine, asparagine, glutamine, tyrosine, cysteine, lysine, histidine, aspartic acid, or glutamic acid.

b) The polynucleotide encodes an amino acid other than leucine at position 425 of SEQ ID NO: 2; at position 415 of SEQ ID NO: 4; at position 413 of SEQ ID NO: 6; or at position 409 of SEQ ID NO: 8. Illustratively, the amino acid can be proline.

c) The polynucleotide encodes an amino acid other than valine at position 509 of SEQ ID NO: 2; at position 499 of SEQ ID NO: 4; at position 497 of SEQ ID NO: 6; or at position 493 of SEQ ID NO: 8. Illustratively, the amino acid can be glycine.

d) The polynucleotide encodes an amino acid other than leucine at position 542 of SEQ ID NO: 2; at position 532 of SEQ ID NO: 4; at position 530 of SEQ ID NO: 6; or at position 526 of SEQ ID NO: 8. Illustratively, the amino acid can be arginine.

Herbicide resistance may be achieved by any one of the above described amino acid substitutions and by combinations thereof. Further, standard testing may be used to determine the level of resistance provided by the various mutations or combinations thereof, and the level of wild-type catalytic activity (if any) retained by the enzyme.

Another preferred set of polynucleotides of the invention includes those that encode an entire plant PDS precursor protein (including the mature protein and a transit peptide), the protein having one or more amino acid changes due to point mutations providing an increase in bleaching herbicide resistance as discussed above. Accordingly, additional preferred polynucleotides are provided wherein they encode a plant PDS precursor protein having an amino acid sequence at least 80% identical to the entirety of any one of SEQ ID NOs: 2, 4, 6, and 8, the precursor protein having one or more point mutations as discussed herein. More preferably, such polynucleotides encode a plant precursor protein having an amino acid sequence at least 90% identical to any one of SEQ ID NOs: 2, 4, 6, and 8, and most preferably at least 95% identical.

Another set of preferred polynucleotides of the invention are those that encode a mutant plant phytoene desaturase enzyme with increased resistance to one or more bleaching herbicides, wherein the polynucleotides have a nucleotide sequence at least about 60% identical to nucleotides 324 to 1748 of SEQ ID NO: 1, nucleotides 509 to 1933 of SEQ ID NO: 3, nucleotides 633 to 2066 of SEQ ID NO: 5, or nucleotides 275 to 1705 of SEQ ID NO: 7. More preferably, such polynucleotides have a nucleotide sequence at least about 90% identical to any one of the above-identified nucleotide ranges/SEQ ID's, and most preferably at least about 95% identical. Preferably, these polynucleotides encode mutant phytoene desaturase enzymes having one or more amino acid point mutations as discussed above. These polynucleotides are expected to code for mutant PDS precursor proteins that include chloroplast transit peptides that will target the proteins to chloroplasts when expressed after nuclear transformation with the polynucleotides. On the other hand, where the mutant plant PDS-encoding polynucleotides of the invention are incorporated into the plastidic genome of plants, the use of such transit peptides is expected to be unnecessary, and polynucleotides encoding only for the mature mutant plant PDS proteins may be used.

Polynucleotides of the invention can be prepared, for example, by obtaining or isolating a wild-type PDS gene from a plant species of interest, and introducing the desired mutation by site-directed mutagenesis. For example, such mutations can be introduced via directed mutagenesis techniques such as homologous recombination. Illustratively, the amino acid substitution(s) required for herbicide resistance can be achieved by mutating a polynucleotide encoding a herbicide sensitive PDS from any plant of interest generally as follows:

(1) isolate genomic DNA or mRNA from the plant;

(2) prepare a genomic library from the isolated DNA or a cDNA library from the isolated RNA;

(3) identify those phages or plasmids which contain a DNA fragment encoding PDS;

(4) sequence the fragment encoding the PDS;

(5) sub-clone the DNA fragment carrying the PDS gene into a cloning vehicle which is capable of producing single-stranded DNA;

(6) synthesize an oligonucleotide of about 15 to 20 nucleotides which is complementary to a particular PDS nucleotide sequence encoding one of the amino acid sub-sequences recited above except for the nucleotide change(s) required to direct a mutation to a codon for an amino acid selected for its ability to confer herbicide resistance;

(7) anneal the oligonucleotide to the single-stranded DNA containing the region to be mutated and use it to prime synthesis in vitro of a complementary DNA strand forming a heteroduplex;

(8) transform bacterial cells with the heteroduplex DNA;

(9) screen the transformed bacterial cells for those cells which contain the mutated DNA fragment by a) immobilizing the DNA on a nitrocellulose filter, b) hybridizing it to the 5′-32 P labeled mutagenic oligonucleotide at ambient temperature, and c) washing it under conditions of increasing temperature so as to selectively dissociate the probe from the wild-type gene but not the mutant gene;

(10) isolate a double-stranded DNA fragment containing the mutation from the cells carrying the mutant gene; and

(11) confirm the presence of the mutation by DNA sequence analysis.

An amino acid substitution required for herbicide resistance can also be achieved by substituting a nucleotide sequence of a plant PDS gene which encodes a sequence of amino acids containing the amino acid to be substituted with another nucleotide sequence, which encodes the corresponding stretch of amino acids containing the desired substitution, derived from any natural PDS gene or from a synthetic source.

Preferred nucleic acid constructs of the invention will include an inventive mutant plant PDS polynucleotide and at least one regulatory nucleotide sequence. For example, nucleic acid constructs of the invention will typically include the mutant plant PDS polynucleotide in operable association with a promoter, such as a constitutive or other promoter effective to provide sufficient expression of the mutant plant PDS polynucleotide in a plant, plant cell or plant tissue to confer bleaching herbicide resistance. Nucleic acid constructs of the invention may, for example, be in the form of a vector such as a plasmid, virus or cosmid that contains the mutant plant PDS polynucleotide.

Particularly preferred nucleic acid constructs of the invention will include a polynucleotide encoding a mutant PDS precursor protein having a chloroplast transit peptide and a resistant PDS protein of the invention, wherein the polynucleotide is under expression control of a plant operable promoter. In such constructs, the promoter can be heterologous or non-heterologous (native) with respect to the polynucleotide, and the chloroplast transit peptide can be heterologous or non-heterologous (native) with respect to the PDS protein. In preferred forms, both the promoter and the transit peptide will be native to the PDS enzyme. For example, the transit peptide, and the nucleotide sequence encoding it, may be any one of those identified in SEQ ID Nos 1-8.

A preferred nucleic acid construct will thus include the following components in the 5′ to 3′ direction of transcription:

(i) a plant operable promoter;

(ii) a genomic sequence which encodes a chloroplast transit peptide;

(iii) a nucleotide sequence (including a genomic sequence) which encodes a resistant mutant plant PDS protein as described herein; and

(iv) a transcriptional terminator.

The polynucleotides and nucleic acid constructs of the present invention can be used to introduce herbicide resistance into plants. A wide variety of known techniques for this purpose may be used, and will differ depending on the species or cultivar desired. For example, in respect of the transformation of plant material, those skilled in the art will recognize that both the target material and the method of transformation (e. g. Agrobacterium or particle bombardment) can be varied. In some general transformation protocols, explants or protoplasts can be taken or produced from either in vitro or soil grown plants. Explants or protoplasts may be produced from cotyledons, stems, petioles, leaves, roots, immature embryos, hypocotyls, inflorescences, etc. In theory, any tissue which can be manipulated in vitro to give rise to new callus or organized tissue growth can be used for this genetic transformation. Plant organs that may be used include but are not limited to leaves, stems, roots, vegetative buds, floral buds, meristems, embryos, cotyledons, endosperm, sepals, petals, pistils, carpels, stamens, anthers, microspores, pollen, pollen tubes, ovules, ovaries and fruits, or sections, slices or discs taken therefrom. Plant tissues that may be used include, but are not limited to, callus tissues, ground tissues, vascular tissues, storage tissues, meristematic tissues, leaf tissues, shoot tissues, root tissues, gall tissues, plant tumor tissues, and reproductive tissues. Plant cells include, but are not limited to, isolated cells with cell walls, variously sized aggregates thereof, and protoplasts.

To achieve transformation, explants or protoplasts may be cocultured with Agrobacterium, which can be induced to transfer polynucleotides located between the T-DNA borders of the Ti plasmid to the plant cells. These explants can be cultured to permit callus growth. The callus can then be tested directly for resistance to PDS inhibiting herbicides, or plants can be regenerated and the plants tested for herbicide resistance. Such testing may include an enzyme assay of plant cell extracts for the presence of PDS activity resistant to herbicide and/or growth of plant cells in culture or of whole plants in the presence of normally inhibitory concentrations of herbicide. Another transformation method is direct DNA uptake by plant protoplasts. With this method, the use of Agrobacterium is bypassed and DNA is taken up directly by the protoplasts under the appropriate conditions.

Nucleic acid constructs of the invention can thus be derived from a bacterial plasmid or phage, from the Ti- or Ri-plasmids, from a plant virus or from an autonomously replicating sequence. Preferred nucleic acid constructs will be derived from Agrobacterium tumefaciens containing the mutant plant PDS-encoding polynucleotide of the invention between T-DNA borders either on a disarmed Ti-plasmid (a Ti-plasmid from which the genes for tumorigenicity have been deleted) or in a binary vector in trans to a Ti-plasmid with Vir functions. The Agrobacterium can be used to transform plants by inoculation of tissue explants, such as stems or leaf discs, or by co-cultivation with plant protoplasts, as noted above.

Another preferred means of introducing the polynucleotides involves direct introduction of the polynucleotide or a nucleic acid construct containing the polynucleotide into plant protoplasts or cells, with or without the aid of electroporation, polyethylene glycol or other agents or processes known to alter membrane permeability to macromolecules.

The polynucleotides and nucleic acid constructs of the invention can be used to transform a wide range of higher plant species to form plants of the present invention. The plant can be of any species of dicotyledonous, monocotyledonous or gymnospermous plant, including any woody plant species that grows as a tree or shrub, any herbaceous species, or any species that produces edible fruits, seeds or vegetables, or any species that produces colorful or aromatic flowers. For example, the plant may be selected from a species of plant from the group consisting of canola, sunflower, tobacco, sugar beet, cotton, maize, wheat, barley, rice, sorghum, tomato, mango, peach, apple, pear, strawberry, banana, melon, potato, carrot, lettuce, onion, soya spp, sugar cane, pea, field beans, poplar, grape, citrus, alfalfa, rye, oats, turf and forage grasses, flax, oilseed rape, cucumber, morning glory, balsam, pepper, eggplant, marigold, lotus, cabbage, daisy, carnation, tulip, iris, lily, and nut producing plants insofar as they are not already specifically mentioned. Particularly preferred are crop plants, especially maize, soybean, rice, cotton, wheat, canola, and tobacco.

One could further increase the level of expression of the polynucleotides of the invention by replacing their native regulatory nucleotide sequences, 5′ and 3′ to the PDS coding sequence, with synthetic or natural sequences known to provide high level and/or tissue specific expression. One may also substitute the nucleotide sequences of the polynucleotides of the invention with other synthetic or natural sequences which encode transit peptides which will allow efficient chloroplast uptake of the polynucleotides of the invention.

The polynucleotides and nucleic acid constructs of the present invention also have utility as selectable markers for both plant genetic studies and plant cell transformations. A gene of interest, generally conferring some agronomically useful trait, e.g. disease resistance, resistance to insects, fungi, viruses, bacteria, nematodes, stress, dessication, and herbicides, can be introduced into a population of sensitive plant cells physically linked to a polynucleotide of the present invention (e.g. on the same nucleic acid construct). Cells can then be grown in a medium containing a herbicide to which the PDS encoded by a polynucleotide of the invention is resistant. The surviving (transformed) cells are presumed to have acquired not only the herbicide resistance phenotype, but also the phenotype conferred by the gene of interest. The polynucleotides can be introduced by cloning vehicles, such as phages and plasmids, plant viruses, and by direct nucleic acid introduction. Subsequently, in a plant breeding program, the agronomically useful trait can be introduced into various cultivars through standard genetic crosses, by following the easily assayed herbicide resistance phenotype associated with the linked selectable genetic marker.

Illustratively, genes providing insecticidal proteins may be selected from the group consisting of crystal toxins derived from Bt, including secreted Bt toxins; protease inhibitors, lectins, Xenhorabdus/Photorhabdus toxins, with some specific insecticidal proteins including cryIAc, cryIAb, cry3A, Vip 1A, Vip 1B, cystein protease inhibitors, and snowdrop lectin. Fungus resistance conferring genes may be selected from the group consisting of those encoding known AFPs, defensins, chitinases, glucanases, and Avr-Cf9. Illustrative bacterial resistance conferring genes include those encoding cecropins and techyplesin and analogues thereof. Virus resistance conferring genes include for example those encoding virus coat .proteins, movement proteins, viral replicases, and anti-sense and ribozyme sequences which are known to provide for virus resistance. Illustrative stress, salt, and drought resistance conferring genes include those that encode Glutathione-S-transferase and peroxidase, the sequence which constitutes the known CBF1 regulatory sequence and genes which are known to provide for accumulation of trehalose.

Another aspect of the present invention is directed to a non-transgenic plant or plant cell having one or more mutations in the PDS gene, which plant or cell has increased resistance to at least one bleaching herbicide, and which plant exhibits substantially normal growth or development of the plant, its organs, tissues or cells, as compared to the corresponding wild-type plant or cell.

A nontransgenic plant having a mutated PDS gene that substantially maintains the catalytic activity of the wild-type protein irrespective of the presence or absence of a bleaching herbicide can be prepared by known targeted mutagenesis techniques that involve introducing into a plant cell or tissue a recombinogenic oligonucleotide with a targeted mutation in the PDS gene and thereafter identifying a derived cell, seed, or plant having a mutated PDS gene. The recombinagenic oligonucleotide can be introduced into a plant cell or tissue using any method commonly used in the art, including but not limited to, microcarriers (biolistic delivery), microfibers, electroporation, microinjection.

Non-transgenic plants having in their genome a mutated PDS gene as described herein may also be produced using random mutagenic breeding techniques and subsequent selection of resistant varieties. For example, tissue culture cells or seeds can be subjected to physical or chemical mutagenic agents and subsequently selected for PDS-inhibiting herbicide resistance. Mutagenic agents useful for these purposes include for example physical mutagens such as X-rays, gamma rays, fast or thermal neutrons, protons, and chemical mutagens such as ethyl methane sulfonate (EMS), diethyl sulfate (DES), ethylene imine (EI), propane sulfone, N-methyl-N-nitroso urethane Map, nitrosomethyl urea (NMU), ethylnitrosourea (ENU), and other chemical mutagens.

Another aspect of the invention provides methods for controlling the growth of unwanted vegetation occurring in a cultivated area where desired, bleaching herbicide-resistant plants (preferably a crop plant such as maize, soybean, rice or tobacco) of the invention are growing. In these methods, an effective amount of a bleaching herbicide to which the desired plants are resistant is applied to the area, so as to kill the unwanted vegetation but have substantially no deleterious effect on the desired plants. In such methods, the bleaching herbicide may be applied alone or in combination to the area, pre- and/or post-emergence.

The polynucleotides, nucleic acid constructs, and cells, tissues or organisms (e.g. plants) transformed to contain them, also have utility in screening for additional bleaching herbicide compounds that may be effective against mutants resistant to known bleaching herbicides. For example, in vitro assays, including rapid throughput cellular or non-cellular enzyme/substrate based assays, can be developed for these purposes.

For the purpose of promoting a further understanding of the present invention and its features and advantages, the following specific Examples are provided. It will be understood that these Examples are illustrative, and not limiting, of the invention.

### EXAMPLE 1

### Isolation of Partial Hydrilla-PDS cDNA

The following abbreviations apply:
Y=C+T
R=A+G
W=A+T
B=G+T+C
N=A+C+G+T

Based on an alignment with publicly available PDS-sequences (maize #U37285, rice #AF049356, tomato #X59948, soybean #M64704) degenerative primers were designed in suitable regions where the nucleotide-sequence was conserved between the species. The PCR primer pair PDS-819 (5′-TAA AYC CTG ATG ARY TWT CAN TGC-3′) and RPDS-1219 (5′-GTG TTB TTC AGT TTT CTR TCA A-3′) (numbers are based on there position in the nucleotide-sequence of Oryza sativa, Accession #AF049356), were used to yield a PCR fragment of approximately 400 bp.

Total RNA was extracted from Hydrilla leaves with the RNeasy Plant Mini Kit (Qiagen; Cat #74106), according to the manufacturer's protocol, except the washing step with buffer RW1 was done twice, each with 700 μl.

A 400-bp fragment located in the middle of the PDS-gene was amplified with the degenerated primer pair PDS-819 and RPDS-1219, using Hydrilla-total RNA and the GeneAmp EZ rTth RNA PCR Kit (Perkin Elmer Part No. N808-0179) as follows: In a 200 μl MicroAmp reaction tube (PE Biosystems, CA; Part No. N801-0580, with MicroAmp caps Part No. N801-0535), 9.5 μl DEPC-treated ddH₂O, 5 μl 5× EZ-buffer, 3 μl dNTP-mix (2.5 mM each), 0.75 μl of each primers PDS-819 and RPDS-1219 (15 μM each), 1 μl rTth-polymerase (2.5 U/μl) and 2.5 μl total RNA were combined on ice to a total volume of 25 μl and incubated in a Gene Amp PCR System 9700 thermal cycler (PE Applied Biosystems) thermal cycler for one initial cycle for 30 min at 60° C. and 60 sec at 94° C. followed by 40 cycles of 30 sec at 94° C., 30 sec at 60° C. and 60 sec at 72° C. The reaction was completed by a 7 min incubation at 72° C. and cooling to 4° C. The reaction was analyzed by TAE-agarose-gel electrophoresis (1.2% Agarose, 5 V per cm, 40 min). DNA was visualized by UV-light and the 400-bp band was cut out of the gel with a razor blade.

The 400-bp fragment was isolated out of the agarose using the Qiaquick Gel Extraction Kit (Qiagen Inc, CA; #28704), following the manufacturer's instructions. The purified 400-bp fragment was cloned into TOP10-E. coli cells using the TOPO TA (plasmid vector) Cloning Kit (Invitrogen Inc., CA, Cat. #K4575-01), according to the manufacturers protocol. Four of the resulting bacterial colonies were grown overnight in LB-medium (10 g/L peptone from caseine, 5 g/L yeast extract, 10 g/L sodium chloride, pH 7) and plasmid DNA was extracted the following morning using the Qiaprep Spin Miniprep Kit (#27104, Qiagen Inc., CA) according to the manufacturer's protocol. Both strands of the inserts were sequenced using a LiCOR 4200 sequencer using the manufacture's protocol for labeled M13 Forward (#4200-20, M13-Forward (−29)/IRD700 dye labeled primer, 5′-CAC GAC GTT GTA AAA CGA C-3′) and Reverse primers (LiCOR Inc. #40000-21B, M13 Reverse/IRD800 dye-labeled primer, 5′-GGA TAA CAA TTT CAC ACA GG-3′) from LiCOR Inc., Nebrasca.

Resulting sequence information was assembled and analyzed with the Seqman-module of the Lasergene package (DNASTAR, Inc., WI). Based on this sequence information, new primerswere designed using PrimerSelect (Lasergene Inc.) for RACE-experiments (determination of the 5′ and 3′ regions of coding region).

### EXAMPLE 2

### RACE (Rapid Amplification of cDNA Ends)

To obtain the sequence of the complete Hydrilla PDS coding region, 3′- as well as a 5′-RACE were performed with the SMART RACE cDNA Amplification Kit (Clontech, Catalog #K1811-1). Total RNA was used (extracted as described above) and cDNA (3′- and 5′-ready cDNA) was synthesized according to the manufacturer's protocol. 3′-RACE-PCR was performed using the 3′-ready cDNA and the primers UPM (provided in kit) and PDS-1 (5′-TAA AYC CTG ATG AGY TWT CGA TGC AAT G-3′), 5′-RACE was performed using the primers UPM (provided in kit) and RPDS-400 (5′-GTG TTG TTC AGT TTT CTG TCA AAC C-3′) according to the manufacturer's protocol using the “touchdown-PCR” thermal cycler conditions. Agarose gel electrophoresis showed a distinct band for the 3′-RACE at about 1,000 bp, which was cut out of the gel. Because the 5′-RACE failed to give a specific product, 5 μl of the primary PCR product was diluted into 245 μl of Tricine-EDTA. 5 μl of this dilution was used for a nested PCR-reaction with the primers NUP (provided in kit) and RPDS-153 (5′-GGC CAC CCA ATG ACT CGA TGY GAT CAG C-3′). Cycling conditions were 20 cycles of 94° C. for 5 sec, 65° C. for 10 sec and 72° C. for 3 min. The PCR product was used in agarose gel electrophoresis and revealed in a distinct band of about 900-bp. This band was cut out of, the gel.

The specific fragments of the 3′ and 5′-RACE were extracted from the agarose gel using the Qiaquick Gel extraction Kit according to the manufacturer's protocol. The purified fragments were cloned into TOP10-cells with the TOPO TA Cloning Kit (Invitrogen Inc., CA, Cat. #K4575-01). Resulting colonies were grown overnight in LB-medium with kanamycin and extracted the following morning with Qiaprep Spin Miniprep Kit according to the manufacturers protocol and sequenced as previously described. Resulting sequences were analyzed with Seqman and since the sequences of the 3′ and 5′ RACE were overlapping, they were assembled to produce the whole hydrilla PDS sequence. Based on this sequence information, PCR-primers were designed to amplify and clone the coding region as one unit from various hydrilla biotypes.

### EXAMPLE 3

### Amplification of PDS-Gene From Different Hydrilla Biotypes

Total RNA was extracted from frozen hydrilla leaves as described above. For cDNA-synthesis, 2 μg Hydrilla-total RNA, 500 ng Oligo (dT)₁₂₋₁₈ (Invitrogen Inc., CA, Cat. #N420-01) and DEPC-treated water were combined to a volume of 12 μl in a 200 μl MicroAmp-tube. The reaction was placed in a thermal cycler (Perkin-Elmer GeneAmp System 9700) and incubated for 10 minutes at 70° C. The incubation was followed by a quick chill on ice. Additional components (4 μl First Strand Buffer (Life Technologies; Cat #18064-014), 2 μl 0.1 M DTT, 1 μl 10 mM dNTP-mix and 1 μl SuperScript Reverse Transcriptase (200 U/μl) (Life Technologies; Cat #18064-014) were added on ice followed by an incubation at 42° C. for 52 min in a thermal cycler (PE 9700). The reaction was stopped after 52 min by a 10 min incubation at 70° C. and cooled to 4° C.

The cDNA was used as template in a PCR with the components of the Advantage-HF 2 PCR Kit (Clontech Inc., CA, Cat. #K1914-1) and the primer pair ORF-primer: (5′-ATG ACT GTT GCT AGG TCG GTC GTT-3′) and RPDS-1849 (5′-TAC CCC CTT TGC TTG CTG ATG-3′) in a 200 μl MicroAmp-tube on ice as follows: 15.5 μl PCR-Grade H₂O, 2.5 μl 10× HF 2 PCR Buffer, 2.5 μl 10× HF 2 dNTP-mix, 1 μl of each ORF-primer (10μM) and RPDS-1849 (10 μM), 2 μl of cDNA and 0.5 μl Advantage HF 2 Polymerase Mix. The tubes were capped and incubated in a PE 9700 thermal cycler using the following cycling conditions: 30 cycles of 94° C. for 5 sec, 10 sec for 55° C. and 72° C. for 2 min. After the last cycle the reactions were cooled to 4° C. and stored at −20° C. Reactions were analyzed by TAE-agarose gel electrophoresis. The PCR resulted in a single band at about 1,800-bp. These bands were cut out of the gel, isolated and cloned as described above. The only difference was, that the Zero Blunt TOPO-PCR Cloning Kit (Invitrogen, Cat. #K2875-20) was used to clone the fragments according to the manufacturers protocol, because the Advantage HF 2 Polymerase has proofreading capabilities. Bacterial colonies were grown overnight and plasmids were isolated as described above. Sequencing was performed on the LiCOR 4200 as previously described using the standard M13 primers and internal PDS-sequencing primer (PDS Forward, 5′-CCA ATG GAA ATA TAA TAA CAG GAG-3′ with 5′ IRDye 700 and PDS Reverse, 5′-TTC GGG AAT TAA GGA TGA CT-3′ with 5′ IRDye 800, LiCOR, Inc.) on at least 6 independent clones. In addition, 4 of these clones were also sequenced using BigDye Terminators (Cat. 4390242 Applied Biosystems) on a 3700 sequencer (Applied Biosystems). Plasmid DNA for the 3700 was prepared and sequenced as follows:
1. Centrifuge 1.5-3.0 ml overnight culture in 15 ml centrifuge tube. Decant media, blot on paper towel to remove excess liquid. Add 151 Rnase A stock per ml Solution P1−make this fresh each day.
2. Add 250 μl Solution P1+RnaseA. Vortex mix to resuspend pellet. Add 250 μl Solution P2 and mix gently. Let stand 2 min. or until lysate is clear. Add 350 μl Solution P3 and mix. Add 30 μl Precipitate and mix well. Let stand at room temp 5 min.
3. Centrifuge 10 min at 20,000 g.
4. Remove 800 μl supernatant and transfer to new tube. Add 560 μl isopropanol to filtrate and vortex to mix well. Centrifuge 30 min at 20,000 g. Decant supernatant, wash with 100 μl 70% EtOH. Centrifuge 3 min at 20,000 g. Remove supernatant, air dry briefly and resuspend pellet in 50-100 μl water.
5. Sample plate for DNA concentration on an agarose gel using known plasmid DNA to quantitate. It is important to determine whether there is RNA contamination that will cause underestimation of the DNA template using spectrometry. Use 200 ng per sequencing rxn.
Precipitate Cat. P00050-30 Ligochem 1-973-575-0082
Solution P1 Cat. 19051 (500 ml) Qiagen, Inc.
Solution P2 Cat. 19052 (500 ml) Qiagen, Inc.
Solution P3 Cat. 19053 (500 ml) Qiagen, Inc.
Rnase A Cat. 19101 (250 mg) Qiagen, Inc.
   Plasmid Sequencing with BigDye Per rxn

BigDye terminator mix 0.5 μl, BD buffer 1.75 μl, 8 picomoles of sequencing primer, DNA (in water) 200 ng, Water to 10 μl final volume.

### Cycle Sequencing Conditions:

1=96° C.-2 min
2=96° C.-30 sec
3=50° C.-1 min
4=60° C.-4 min
5=Go to step 2, 24 times
6=4° C.-hold

### Precipitation of Sequencing Reactions and Removal of Unincorporated Dye:

Add 40 μl of precipitation solution to each tube and mix. Let stand at room temp at least 15 minutes (can go up to several hours). Centrifuge at 20,000 g for 30 min at room* temp. Remove supernatant and wash with 100 μl of 70% ethanol. Centrifuge at 20,000 g for 3 min and remove supernatant as above. Air dry 10 min and store at −20 C. Pellet was resuspended in loading buffer and loaded onto ABI 3700 sequencer.

### EXAMPLE 4

### Mutageneis of Arg³⁰⁴

### Non-His-Tagged Phytoene Desaturase Expression Vectors and Transformed Cells

All mutagenesis was performed, unless stated otherwise, using the TOPO-vector containing the wildtype (H4) PDS-sequence, which was extracted from an overnight culture with the Qiagen Plasmid Prep Kit as described above. The amount of the extracted plasmid was diluted to 10 ng/μl and used as template in a mutagenesis-procedure using the QuikChange Site-directed Mutagenesis Kit (Stratagene, Calif., #200518). The design of the mutagenesis-primers was conducted according to the manufacturers protocol to enable the specific change of Arg³⁰⁴-codon to code for the desired amino acid. Primers (purchased from MWG-Biotech) and their introduced amino acid change are listed below with the changed codon underlined:

Alanine :
Hyd-Ala-For
GCATCCTGATTGCCTTAAACGCTTTCCTTCAGGAAAAGC
Hyd-Ala-Rev
GCTTTTCCTGAAGGAAAGCGTTTAAGGCAATCAGGATGC
Asparagine
Hyd-Asn-For
GCATCCTGATTGCCTTAAACAATTTCCTTCAGGAAAAGC
Hyd-Asn-Rev
GCTTTTCCTGAAGGAAATTGTTTAAGGCAATCAGGATGC
Aspartic acid
Hyd-Asp-For
GCATCCTGATTGCCTTAAACGATTTCCTTCAGGAAAAGC
Hyd-Asp-Rev
GCTTTTCCTGAAGGAAATCGTTTAAGGCAATCAGGATGC
Glutamic acid
Hyd-Glu-For
GCATCCTGATTGCCTTAAACGAGTTCCTTCAGGAAAAGC
Hyd-Glu-Rev
GCTTTTCCTGAAGGAACTCGTTTAAGGCAATCAGGATGC
Glutamine
Hyd-Gln-For
GCATCCTGATTGCCTTAAACCAGTTCCTTCAGGAAAAGC
Hyd-Gln-Rev
GCTTTTCCTGAAGGAACTGGTTTAAGGCAATCAGGATGC
Isoleucine
Hyd-Ile-For
GCATCCTGATTGCCTTAAACATTTTCCTTCAGGAAAAGC
Hyd-Ile-Rev
GCTTTTCCTGAAGGAAAATGTTTAAGGCAATCAGGATGC
Lysine
Hyd-Lys-For
GCATCCTGATTGCCTTAAACAAGTTCCTTCAGGAAAAGC
Hyd-Lys-Rev
GCTTTTCCTGAAGGAACTTGTTTAAGGCAATCAGGATGC
Methionine
Hyd-Met-For
GCATCCTGATTGCCTTAAACATGTTCCTTCAGGAAAAGC
Hyd-Met-Rev
GCTTTTCCTGAAGGAACATGTTTAAGGCAATCAGGATGC
Phenylalanine
Hyd-Phe-For
GCATCCTGATTGCCTTAAACTTCTTCCTTCAGGAAAAGC
Hyd-Phe-Rev
GCTTTTCCTGAAGGAAGAAGTTTAAGGCAATCAGGATGC
Threonine
Hyd-Thr-For
GCATCCTGATTGCCTTAAACACTTTCCTTCAGGAAAAGC
Hyd-Thr-Rev
GCTTTTCCTGAAGGAAAGTGTTTAAGGCAATCAGGATGC
Tyrosine
Hyd-Tyr-For
GCATCCTGATTGCCTTAAACTATTTCCTTCAGGAAAAGC
Hyd-Tyr-Rev
GCTTTTCCTGAAGGAAATAGTTTAAGGCAATCAGGATGC
Tryptophan
Hyd-Trp-For
GCATCCTGATTGCCTTAAACTGGTTCCTTCAGGAAAAGC
Hyd-Trp-Rev
GCTTTTCCTGAAGGAACCAGTTTAAGGCAATCAGGATGC
Valine
Hyd-Val-For
GCATCCTGATTGCCTTAAACGTTTTCCTTCAGGAAAAGC
Hyd-Val-Rev
GCTTTTCCTGAAGGAAAACGTTTAAGGCAATCAGGATGC
Glycine
Hyd-Gly-For
GCATCCTGATTGCCTTAAACGGTTTCCTTCAGGAAAAGC
Hyd-Gly-Rev
GCTTTTCCTGAAGGAAACCGTTTAAGGCAATCAGGATGC
Histidine
Hyd-His-For
GCATCCTGATTGCCTTAAACCATTTCCTTCAGGAAAAGC
Hyd-His-Rev
GCTTTTCCTGAAGGAAATGGTTTAAGGCAATCAGGATGC
Leucine
Hyd-Leu-For
GCATCCTGATTGCCTTAAACCTTTTCCTTCAGGAAAAGC
Hyd-Leu-Rev
GCTTTTCCTGAAGGAAAAGGTTTAAGGCAATCAGGATGC
Proline
Hyd-Pro-For
GCATCCTGATTGCCTTAAACCCTTTCCTTCAGGAAAAGC
Hyd-Pro-Rev
GCTTTTCCTGAAGGAAAGGGTTTAAGGCAATCAGGATGC
Cysteine
Hyd-Cys-For
GCATCCTGATTGCCTTAAACTGTTTCCTTCAGGAAAAGC
Hyd-Cys-Rev
GCTTTTCCTbAAGGAAACAGTTTAAGGCAATCAGGATGC
Serine
Hyd-Ser-For
GCATCCTGATTGCCTTAAACAGTTTCCTTCAGGAAAAGC
Hyd-Ser-Rev
GCTTTTCCTGAAGGAAACTGTTTAAGGCAATCAGGATGC
Arginine (reversion to wildtype)
Hyd-WT-For
GCATCCTGATTGCCTTAAACCGTTTCCTTCAGGAAAAGC
Hyd-WT-Rev
GCTTTTCCTGAAGGAAACGGTTTAAGGCAATCAGGATGC

The reactions were performed according to the manufacturers protocol. Reactions were set up in MicroAmp-tubes on ice with 38 μl ddH₂O, 5 μl 10× reaction buffer, 2 μl plasmid (10 ng/μl), 1.25 μl forward-mutagenesis primer (100 ng/μl), 1.25 μl of reverse mutagenesis primer (100 ng/μl), 1 μl dNTP-mix and 1 μl PfuTurboDNA polymerase (2.5 U/μl). The reactions were placed in a PE 9700 thermal cycler and heated to 95° C. for 30 sec followed by 12 rounds at 95° C. for 30 sec, 55° C. for 1 min and 68° C. for 12 min. The PCR was followed by a DpnI-digestion and transformation in XL1-Blue supercompetent cells as described in the manual. 4-6 resulting colonies were grown overnight in LB-medium with kanamycin and plasmid DNA was isolated as described above. The plasmid was used as template for sequencing with M13 and internal PDS-primers as described above on a LiCOR-system. Sequences were assembled and analyzed using Seqman. Introduced mutations were identified and plasmids carrying the desired mutation(s) were transferred into competent TOP10-cells, using the transformation protocol from the TOPO TA Cloning Kit. Resulting colonies were grown overnight in Wu-broth with kanamycin, aliquoted and stored at −80° C. until further use. 1 ml of Wu-cultures was used to start 1-L LB-cultures with kanamycin as described before, to express active PDS-enzyme for testing as described.

### His-Tagged Phytoene Desaturase Bacterial Expression Vectors and Cell Transformation

The plasmid pHy4ATG5 was made by cloning the Phytoene Desaturase (pds) gene from Hydrilla verticillata, including 323 by upstream of the beginning of the putative mature protein, into the vector TOPO4 (Invitrogen, Carlsbad, Calif.): The 1-323 by region contained three potential start codons (ATG)(positions 1, 114 and 225 bp) in frame with pds. In order to express pds in bacteria, deletion clones were made for each of the three potential start codons with and without ATG. Only the results for possible origins of translation 1 and 225 by (named ORF and 3ORF) are reported here. pds was PCR amplified and subcloned into TOPO4 using pHy4ATG5 as template and the reverse primer RPDS_{—}1849 (5′taccccctttgcttgctgatg 3′). The forward primers used were ORF (5′ atgactgttgctaggtcggtcgtt 3′), ORF-ATG (5′ actgttgctaggtcggtcgttgc 3′), 3_ORF (5′atggatttcccaagacctgatatag 3′) and 3_ORF-ATG (5′ gatttcccaagacctgatatagataac 3′). The resulting plasmids were named pORF, pORF-ATG (minus ATG codon), p3ORF and p3ORF-ATG. The pds-containing EcoRI-fragments of these plasmids were subcloned into the EcoRI site of pRSETb vector (Invitrogen) for Histidine tagging and bacterial expression. The resulting constructs were pHy4SET, pHy4SET-ATG (minus ATG), p3ORFSET and p3ORFSET-ATG.

Plasmid p3ORF-ATG was later mutagenized at the amino acid 304 of pds to replace the amino acid Arginine (Arg) by Histidine (His), Threonine (Thr), Serine (Ser), or Cysteine (Cys), using the QuickChange™ Site-Directed Mutagenesis Kit of Stratagene (La Jolla, Calif.). Primers used to replace Arg by His were Hyd-His-For (5′gcatcctgattgccttaaaccatttccttcaggaaaagc 3′) and Hyd-His-Rev (5′ gcttttcctgaaggaaatggtttaaggcaatcaggatgc 3′); to replace Arg by Thr we used primers Hyd-Thr-For (5′gcatcctgattgccttaaacactttccttcaggaaaagc 3′) and Hyd-Thr-Rev (5′gcttttcctgaaggaaagtgtttaaggcaatcaggatgc 3′); to replace Arg by Ser we used Hyd-Ser-For (5′gcatcctgattgccttaaacagtttccttcaggaaaagc 3′) and Hyd-Ser-Rev (5′gcttttcctgaaggaaactgtttaaggcaatcaggatgc 3′); and Arg was replaced by Cys using Hyd-Cys-For (5′gcatcctgattgccttaaactgtttccttcaggaaaagc 3′) and Hyd-Cys-Rev (5′gcttttcctgaaggaaacagtttaaggcaatcaggatgc 3′). Resulting plasmids were named: p3ORFHisSet-ATG, p3ORFThrSet-ATG, p3ORFSerSet-ATG and p3ORFCysSet-ATG.

All pRSET derived constructs were transformed into BL21(DE3)pLysS cells (Invitrogen), induced with IPTG for protein expression, and the His tag overproduced proteins analyzed by Western blot using Anti-HisG antibody (Invitrogen, cat. #R940-25).

### EXAMPLE 5

### Testing of Hydrilla PDS Mutants for Fluridone Resistance and Cross-Resistance to Other Herbicides

### A. Preparation of Arg³⁰⁴ Mutant Protein Compositions.

### Non-His-tagged Protein Preparation

Phytoene desaturase activity and its inhibition by herbicides was determined using an in vitro system using components derived from the in vivo production of phytoene and phytoene desaturase proteins. All mutations described in Examples 3 and 4 were tested. Clones from Example 3 were chosen based on the sequencing results with their insert in the correct orientation and with expression driven by the lac prothoter. The clones were used for the heterologous expression of Hydrilla-PDS-enzyme. In particular, bacterial cultures were grown from single colonies overnight in Wu-broth (6.27 g/L K₂HPO₄, 1.8 g/L KH₂PO₄, 0.5 g/L Na-citrate, 0.9 g/L (NH₄)₂SO₄, 10 g/L tryptone, 5 g/L yeast extract, 10 g/L NaCl, 44 ml/L glycerol, 0.1 mM MgSO₄, pH 7.2) with kanamycin. 1-mL was aliquoted in centrifuge tube and stored at −80° C. For expression, 1 L LB-medium with kanamycin was inoculated with 1-mL Wu-culture and grown for 24 h at 37° C. to stationary phase with shaking at 200 rpm. The cells were collected by centrifugation at 2000×g. All of the following steps were done at 4° C. unless otherwise noted. Active soluble PDS enzyme was extracted by lysing the transformed E. coli cells using a French Press at 20,000 psi in assay buffer consisting of 100 mM Tris-HCl, pH 7.2, 10 mM magnesium-chloride, 0.1 mM NADP, 0.1 mM FAD, 10 mM cysteine, 5 mM DTT, 1 mM aminocaproic acid, and 1 μg/ml leupeptin. The soluble fraction containing PDS activity was obtained by centrifugation for 10 min at 1200×g. After 24 h they were centrifuged down and used in PDS enzyme activity assays as described below.

### His-Tagged Protein Preparation

When working with His-tagged PDS proteins expressed as described in Example 4, the purified protein used for testing was prepared as follows. BL21(DE3)pLysS cells were grown overnight in 500 ml Luria Broth (LB) supplemented with carbenicillin (100 mg/l) and cloramphenicol (60 mg/l) at 37° C., and induced with 0.3 mM isopropylthio-β-D-galactoside (IPTG) for 3 hrs. Cells were lysed using a French press (Spectronics Instrument) at 20,000 psi and overexpressed PDS was purified on a nickel activated Hitrap Chelating HP column according to the manufacturers instructions (Amersham Bioscience).

### B. Enzyme Activity Assays for Arg³⁰⁴ Mutant Proteins.

Generally, PDS protein fractions prepared as described above (Non-His-tagged and His-tagged) were mixed with a soluble fraction from phytoene-producing E. coli cells. For the phytoene producing cells, a plasmid construct containing the genes GGPP synthase and phytoene synthase from Erwinia uredovora, transformed into the appropriate E. coli strain, was used (see, Misawa et al., J. of Bacteriology 177:6575, 1995). This E. coli strain was separately cultured and lysed, and its soluble components collected (“the EB extract”) in a fashion similar to that described above for the PDS-producing cells.

### Non-His-Tagged Protein Experiments

Reactions were set up by adding 500 μl of the extract of the various PDS clones, 500 μl of EB extract (containing phytoene) and 5 μl of 10 mM plastoquinone in a 1500 μl microfuge tube. For determining the effect of herbicide on the activity of PDS the appropriate amount of herbicide for activity (Fluridone, Norflurazon, Diflufenican, Picolinafen, Flurtamone, Flurochloridone, or Beflubutamid) was added to the 500 μl of PDS extract and incubated on ice for 15 minutes prior to mixing it with the EB extract. The herbicide concentrations tested ranged from 0.1 nM to 1000 μM; for Fluridone the addition of 10 μL in MeOH was generally used. At the end of the incubation period, the carotenoids were extracted in the dark as follows. The 1 ml reactions were transferred by pipette into 15 ml falcon tubes containing 5 ml of 6% KOH in MeOH to which 4 ml of 10% diethyl ether in benzin was added to the tubes. 2.5 ml of saturated NaCl was added to help in the separation of the phases. The top ether layers were transferred to test tubes, dried under nitrogen gas, and the residue dissolved in 150 μl of acetone. Samples were analyzed by HPLC under the following conditions.

The HPLC system consisted of a Waters Associates (Milford, Mass. 01757, USA) components, which includes a Model 510, pump, a Model 712 autosampler, a Millenium 2010 controller and Models 470 fluorescence and 990 photodiode spectrophotometric detectors. The column was 15 cm×4.6 mm 3 μM Supelcosil LC-18 reversed phase column (Supelco). The solvent system was an isocratic mixture of 50% acetonitrile, 45% 2-propanol, 5% methanol. The samples were injected in 50 μl volume, with a run time of 10 minutes. Carotenoids were detected at 400 nm and phytoene was detected at 287 nm. The results of this testing are set forth in Tables 1 and 2 in the Description above.

### His-Tagged Protein Experiments

His-tagged, purified proteins prepared as described above were transferred to the assay buffer on a PD10 column (Amersham Bioscience) and the concentration was adjusted to 100 μg/mL. Crude extracts containing phytoene were produced in E. coli JM101/pACCRT-EB containing geranylgeranyl pyrophosphate synthase and phytoene synthase enzymes from Erwinia uredova as described above. The reaction assays consisted of 50 μg PDS in 500 μl of assay buffer (200 mM Sodium Phosphate, pH 7.2) and 500 μl of pACCRT-EB extract. The herbicide (10 μL in MeOH) was added to the 500 μl of PDS extract and incubated on ice for 15 minutes prior to mixing it with the EB extract. The assay was carried out for 30 min at 30° C. and 350 rpm on a Eppendorf ThermoMixer-R (Brinkmann Instruments). ζ-Carotene produced was extracted and quantified spectrophotometrically at A₄₂₅ using a extinction coefficient (mM) εₘₐₓ 138. Dose-response curves were fitted to the four-parameter logistic function. However, the equation was simplified to the following since minimum and maximum values were 0 and 100, respectively. I⁵⁰ values were calculated from the regressions.

$$f = \frac{100}{1 + {e}^{b * ( ln ( x ) - ln ( I 50 ) )}}$$

The results are shown in Table 2A in the Description above.

### EXAMPLE 6

### Mutagenesis of Leu⁴²⁵, Val⁵⁰⁹, and Leu⁵⁴² (SEQ. #2)

To test amino acids that were identified to lead to resistance in Cyanobacteria (Synechococcus PCC7942, Synechocystis PCC6803, summary in: G Sandmann, N Misawa, P Böger, Steps towards genetic engineering of crops resistant to bleaching herbicides. 189-200, 1996) the following mutations were introduced in hydrilla-PDS (wildtype) at the position indicated. The procedures for mutagenesis and testing for activity were the same as described in the Examples above. The mutagenesis primers used were designed as follows:

Position 425 leucine (CTT) -> proline (CCT)
Hyd-320-Pro-For
GGAAGTTGAAGAACACATACGATCATCCTCTTTTCAGCAGG
Hyd-320-Pro-Rev
CCTGCTGAAAAGAGGATGATCGTATGTGTTCTTCAACTTCC
Position 509 valine (GTT) -> glycine (GGT)
Hyd-403-Gly-For
GTTGTAAAGACCCCGAGGTCAGGTTACAAGACGGTCC
Hyd-403-Gly-Rev
GGACCGTCTTGTAACCTGACCTCGGGGTCTTTACAAC
Position 542 leucine (TTG) to arginine (AGG)
Hyd-436-Arg-For
GGTGACTACACAAAGCAGAAGTATAGGGCCTCAATGGAAGG
Hyd-436-Arg-Rev
CCTTCCATTGAGGCCCTATACTTCTGCTTTGTGTAGTCACC

The results are shown in Table 3 of the Description above.

### EXAMPLE 7

### Introduction of Mutations at Different Positions

To test for synergism or other effects between multiple mutations at the positions 304, 425, 509 and 542, the same mutagenesis procedure was followed as described above, except that the plasmid already contained an altered amino acid at one location and mutagenesis was performed for a second location. Particularly, the Cys³⁰⁴ with Gly⁵⁰⁹ combination was made and tested. In additional work, all other possible combinations, up to combining mutations on all four different sites, can be created and tested.

### EXAMPLE 8

### Mutagenesis of the Arg-Codon in Corn PDS to His

The same mutagenesis procedure as described in the Examples above was used to convert the same key arginine amino acid (position 292, sequence ID No. 6) to histidine in corn PDS. Mutagenesis primers are listed below. A maize cDNA clone of PDS, that actively expressed PDS (basically sequence U37285 ligated in frame into pBluescript SK⁺ and cloned in TOP10-cells) was provided by Eleanore T. Wurtzel, N Y (pMPDS3-33 as described in Z H Li, P D Matthews, B Burr & E T Wurtzel: Cloning and characterization of a maize cDNA encoding phytoene desaturase, an enzyme of the carotenoid biosynthetic pathway. Plant Molecular Biology 30: 269-279, 1996).

CornMut-For
GCATTTTGATTGCTTTGAACCACTTTCTTCAGGAGAAGC
CornMut-Rev
GCTTCTCCTGAAGAAAGTGGTTCAAAGCAATCAAAATGC

The resulting mutant maize PDS polynucleotide and protein were tested generally as described in Example 5 above. The mutant maize PDS enzyme ehxibited 50-fold to 60-fold resistance factor as compared to the wild type maize PDS enzyme.

### EXAMPLE 9

### Generation of Plants

### Phytoene Desaturase Plant Expression Vectors

Binary vectors for pds expression in plants included the 1-323 by upstream of the beginning of the putative mature protien, which is assumed to encode for chloroplast signal peptide/s. pHy4ATG5 was mutagenized at the amino acid 304 of pds to replace Arginine by Histidine, Threonine, Serine, or Cysteine, using the QuickChange™ Site-Directed Mutagenesis Kit of Stratagene (La Jolla, Calif.) and the same mutagenesis primers used for p3ORF-ATG indicated in the previous section. The resulting plasmids in this case were pHy4His, pHy4Thr, pHy4Ser and pHy4Cys. A 1.8 kb fragment between the TOPO4 SpeI site and the pds SspI site containing the pds gene was cloned into pCAMBIA1303 (CAMBIA, Canberra, Australia) SpeI-Pm1I sites (SspI and PmlI are compatible) replacing the 2.5 kb gus:mgfp; the resulting plasmid was designated pPDATG1303. The same strategy was used for each of the clones containing amino acid changes, generating plasmids pPDHIS1303, pPDTHR1303, pPDSER1303 and pPDCYS1303. The selectable marker in these constructs is the hygromycin phosphotransferase gene (hptll) for resistance to hygromycin in plants.

In order to test for possible differences in pds expression with alternative promoters, the 1.8 kb NcoI-SspI pds fragment from pHy4SET was cloned into the NcoI-PmlI sites of pCAMBIA2301 (CAMBIA, Canberra, Australia). The resulting plasmid was named pPDS-PROM, which has the pds and the nptll (neomycin phosphotransferase II) genes without promoters. Then, the 1.8 kb NcoI fragment from pCAMBIA 2301 was cloned into the NcoI site of pPDS-PROM to add the CaMV35S (35S) and the double CaMV35S (2X35S) promoters to both genes. This resulted in two plasmids, pPDN1X and pPDN2X with pds driven by 35S and 2X35S respectively.

Agrobacterium transformation

The new binary vectors containing pds, as well as pCAMBIA1303 and pCAMBIA2301, were transformed into Agrobacterium tumefaciens strains EHA105 and C58C1 as indicated by Fisher, D. K. and Guiltinan, M. J. (1995) Rapid, efficient production of homozygous transgenic tobacco plants with Agrobacterium tumefaciens: a seed-to-seed protocol. Plant Molecular Biology Report 13(3):278-289. Transformation of Agrobacterium strains was confirmed by plasmid isolation and restriction digestion.

### Plant Transformation

Arabidopsis thaliana ecotype Columbia (Col-0) was transformed with Agrobacterium using the floral dip method (Clough, S. J. and Bent, A. F. (1998) Floral dip: a simplified method for Agrobacterium-mediated transformation of Arabidopsis thaliana. The Plant Journal 16(6):735-743). Plants were grown at 21° C. with 16 h/8 h day and night until flowering and continuous light after inoculation (plants inoculated with Agrobacterium were denominated T0 plants). Selection of T1 (seeds produced by T0 plants) seedlings was performed on Petri plates with half-strength Murashige and Skoog (MS) medium (Murashige, T. and Skoog, F. (1962) A revised medium for rapid grOwth and bioassays with tobacco tissue cultures. Physiologia plantarum 15:473-497) supplemented with 1% sucrose, 0.2% phytagel and 300 μg/ml cefotaxime. Hygromycin or norflurazon was used for selection with pPDATG1303, pPDHIS1303, pPDTHR1303, pPDSER1303 and pPDCYS1303 constructs, while kanamycin was used for pPDN1X and pPDN2X. Agrobacterium strains with pCAMBIA1303, pCAMBIA2301 and without Ti plasmid were used as controls for inoculations. The germination and growing conditions for selection were 24° C. and continuous light.

Nicotiana tabacum cv Xanthi (Smith) was transformed according to Fisher, D. K. and Guiltinan, M. J. (1995) Rapid, efficient production of homozygous transgenic tobacco plants with Agrobacterium tumefaciens: a seed-to-seed protocol. Plant Molecular Biology Report 13(3):278-289. Selection was performed on full strength MS medium supplemented with 3% sucrose, 0.2% phytagel, 400 μg/ml cefotaxime, 100 μg/ml carbenicillin, 1 mg/l Benzylaminopurine, and either kanamycin or hygromycin for selection depending on the construct. Agrobacterium strains with pCAMBIA1303 were used as controls for inoculation. The conditions used for growing and selecting tobacco plants were 25° C. and continuous light.

### Testing for Herbicide Resistance

Arabidopsis T1 seedlings of plants treated with pPDATG1303, pPDTHR1303, pPDSER1303 and pCAMBIA1303 that grew on hygromicin or norflurazon were transferred to half-strength MS medium supplemented with cefotaxime. DNA was extracted according to Dilworth and Frey (2000)foreign genes detected by PCR. Primers used for the detection of Hydrilla PDS were: PDS-START 5′cctcctcaagttgtaattgctggtg 3′ and RPDS-942 5′ttggcttacataatctttcaggtg 3′. Primers used to detect transformation with any of our constructs even without Hydrilla PDS (i.e., pCAMBIA1303, pCAMBIA 2301) were: 2XF 5′agacgtcgcggtgagttcag3′ and 2XR 5′gaggcggtttgcgtattggc3′. From 85 putative transformants selected, 20 were tested by PCR and 18 of them were confirmed as genetically transformed. Of the confirmed transformed plants, 1 of pPDTHR1303, 3 of pPDSER1303 and 1 of pPDATG1303, were resistant to the herbicide norflurazon. Different levels of resistance are expected depending on the construct used. Plants confirmed to be transformants are being cultivated for seed production, and the seeds will be tested for herbicide resistance.

Tobacco plants are at an earlier stage of development, starting to form shoots; those plants will be tested by PCR and propagated in sterile conditions before being tested against fluridone and/or norflurazon.

### EXAMPLE 10

### Generation of Plants: General Methods

Herbicide-resistant plants containing modified PDS genes are generated as follows.

### A. Generation of Vectors for Agrobacterium Transformation

Transformation of Arabidopsis and other plants is commonly achieved with Agrobacterium transformed with a binary vector containing the gene of interest controlled by a desirable promoter. A binary vector is capable of reproducing in E. coli and Agrobacterium, and is more amenable to manipulation through molecular biology protocols. PPZP, pGreen0029, or another suitable vector. The modified PDS genes are cloned downstream of a constitutively expressed promoter (e.g. CaMv 35S) and upstream of a terminator sequence (to stop transcription). This construct is inserted into the selected binary vector. The plasmid DNA from these steps is propagated in E. coli. A liquid culture is then be grown from the “certified” strain and used in the transformation of Arabidopsis and other plants. Agrobacterium-mediated transformation of Arabidopsis and other plants is achieved using known procedures. One such procedure useful for Arabidopsis is the floral dip method as described in Clough and Bent (The Plant J. 16:735, 1998). Briefly, Arabidopsis seedlings are grown to the 2-10 cm stage, where numerous immature floral buds and few siliques are present. These plants are dipped in a solution of Agrobacterium obtained as described above. This method enables the most number of transformed progeny (T0). The transformed seeds are selected by growing them on media containing an antibiotic corresponding to the selectable marker already incorporated in the binary vector. This gives a greater assurance that the plant will contain the resistant PDS gene due to the way the plasmid DNA is inserted into the chromosomal DNA of Arabidopsis. It also provides replications and a reusable seed source. In addition, a.proper level of herbicide resistance may require that the modified PDS gene is homozygous and not heterozygous, as would be in the case of the primary transformants.

Seedlings growing successfully on the selectable media are allowed to mature and produce seeds. This second generation (T1) is tested for resistance to PDS inhibitors by growing them on agar growth media containing various concentrations of PDS inhibitor. The response of wild-type Arabidopsis is standardized for all the PDS inhibitor tested and all successful tranformation are benchmarked to their respective positive controls. Parameters to measure include growth (weight/length) and chlorophyll, carotenoid and phytoene levels. Resistance to fluridone and other PDS inhibitors is then evaluated.

While the invention has been described in detail above with reference to specific embodiments, it will be understood that modifications and alterations in the embodiments disclosed may be made by those practiced in the art without departing from the spirit and scope of the invention. All such modifications and alterations are intended to be covered. In addition, all publications cited herein are indicative of the level of skill in the art and are hereby incorporated by reference in their entirety as if each had been individually incorporated by reference and fully set forth.

1. An isolated polynucleotide containing a nucleic acid sequence encoding a modified plant phytoene desaturase enzyme having increased resistance to one or more bleaching herbicides, the modified plant phytoene desaturase enzyme having at least one amino acid substitution that provides said increased resistance.

2. An isolated polynucleotide according to claim 1, wherein said polynucleotide is selected from:
(a) a polynucleotide encoding a plant phytoene desaturase enzyme having an amino acid sequence at least 80% identical to amino acids 109 to 580 of SEQ ID NO: 2, said amino acid sequence having a point mutation corresponding to one or more of positions 304, 425, 509, and 542 of SEQ ID NO: 2;
(b) a polynucleotide encoding a plant phytoene desaturase enzyme having an amino acid sequence at least 80% identical to amino acids 97 to 570 of SEQ ID NO: 4, said amino acid sequence having a point mutation corresponding to one or more of positions 294, 415, 499, and 532 of SEQ ID NO: 4;
(c) a polynucleotide having encoding a plant phytoene desaturase enzyme having an amino acid sequence at least 80% identical to amino acids 97 to 571 of SEQ ID NO: 6, said amino acid sequence having a point mutation corresponding to one or more of positions 292, 413, 497 and 530 of SEQ ID NO: 6; and
(d) a polynucleotide having encoding a plant phytoene desaturase enzyme having an amino acid sequence at least 80% identical to amino acids 93 to 566 of SEQ ID NO: 8, said amino acid sequence having a point mutation corresponding to one or more of positions 288, 409, 493, and 526 of SEQ ID NO: 8.

3. An isolated polynucleotide according to claim 2, which is a polynucleotide encoding a plant phytoene desaturase enzyme having an amino acid sequence at least 80% identical to amino acids 109 to 580 of SEQ ID NO: 2, said amino acid sequence having a point mutation corresponding to one or more of positions 304, 425, 509, and 542 of SEQ ID NO: 2.

4. An isolated polynucleotide according to claim 3, which encodes a plant phytoene desaturase enzyme that is at least 95% identical to amino acids 109 to 580 of SEQ ID NO: 2.

5. An isolated polynucleotide according to claim 4, which encodes the amino acid sequence from amino acid 109 to 580 of SEQ ID NO: 2, except having a point mutation corresponding to one or more of positions 304, 425, 509, and 542.

6. An isolated polynucleotide according to claim 2, which is a polynucleotide encoding a plant phytoene desaturase enzyme having an amino acid sequence at least 80% identical to amino acids 97 to 570 of SEQ ID NO: 4, said amino acid sequence having a point mutation corresponding to one or more of positions 294, 415, 499, and 532 of SEQ ID NO: 4.

7. An isolated polynucleotide according to claim 6, which encodes an amino acid sequence that is at least 95% identical to amino acids 97 to 570 of SEQ ID NO: 4.

8. An isolated polynucleotide according to claim 7, encodes the amino acid sequence from amino acid 97 to 570 of SEQ ID NO: 4, except having a point mutation corresponding to one or more of positions 294, 415, 499, and 532 of SEQ ID NO: 4.

9. An isolated polynucleotide according to claim 2, which is a polynucleotide encoding a plant phytoene desaturase enzyme having an amino acid sequence at least 80% identical to amino acids 97 to 571 of SEQ ID NO: 6, said amino acid sequence having a point mutation corresponding to one or more of positions 292, 413, 497 and 530 of SEQ ID NO: 6.

10. An isolated polynucleotide according to claim 9, which encodes an amino acid sequence that is at least 95% identical to amino acids 97 to 571 of SEQ ID NO: 6.

11. An isolated polynucleotide according to claim 7, which encodes the amino acid sequence from amino acid 97 to 571 of SEQ ID NO: 6, said amino acid sequence having a point mutation corresponding to one or more of positions 292, 413, 497 and 530 of SEQ ID NO: 6.

12. An isolated polynucleotide according to claim 2, which is a polynucleotide encoding a plant phytoene desaturase enzyme having an amino acid sequence at least 80% identical to amino acids 93 to 566 of SEQ ID NO: 8, said amino acid sequence having a point mutation corresponding to one or more of positions 288, 409, 493, and 526 of SEQ ID NO: 8.

13. An isolated polynucleotide according to claim 12, which encodes an amino acid sequence that is at least 95% identical to amino acids 93 to 566 of SEQ ID NO: 8.

14. An isolated polynucleotide according to claim 13, which encodes the amino acid sequence from amino acids 93 to 566 of SEQ ID NO: 8, said amino acid sequence having a point mutation corresponding to one or more of positions 288, 409, 493, and 526 of SEQ ID NO: 8.

15. A nucleic acid construct comprising a polynucleotide as set forth in claim 1.

16. A nucleic acid construct according to claim 15, wherein said polynucleotide is operably associated with a promoter.

17. A nucleic acid construct according to claim 16, which is an expression vector.

18. An isolated, modified plant phytoene desaturase enzyme having increased resistance to one or more bleaching herbicides, the modified plant phytoene desaturase enzyme having at least one amino acid substitution that provides said increased resistance.

19. An isolated, herbicide-resistant plant phytoene desaturase enzyme according to claim 18, wherein said enzyme has an amino acid sequence at least about 80% identical to any one of SEQ ID NOs. 2, 4, 6, and 8.

20. An herbicide-resistant crop plant including in its genome a polynucleotide containing a nucleic acid sequence encoding a modified plant phytoene desaturase enzyme having increased resistance to one or more bleaching herbicides, the modified plant phytoene desaturase enzyme having at least one amino acid substitution that provides said increased resistance.

21. The herbicide-resistant crop plant of claim 20, wherein said plant is a transgenic plant.

22. The herbicide-resistant crop plant of claim 20, wherein said plant is a non-transgenic plant.

23. The herbicide-resistant crop plant as set forth in claim 20, wherein said crop plant is maize, soybean, or rice.

24. The herbicide-resistant crop plant of claim 23, wherein the crop plant is maize.

25. The herbicide-resistant crop plant of claim 24, wherein the maize plant includes a polynucleotide encoding a modified maize phytoene desaturase enzyme.

26. The herbicide-resistant crop plant of claim 25, wherein the modified maize phytoene desaturase enzyme has an amino acid substitution corresponding to one or more of positions 292, 413, 497 and 530 of SEQ ID NO: 6.

27. The herbicide-resistant crop plant of claim 23, wherein the crop plant is rice.

28. The herbicide-resistant crop plant of claim 27, wherein the rice plant includes a polynucleotide encoding a modified rice phytoene desaturase enzyme.

29. The herbicide-resistant crop plant of claim 28, wherein the modified rice phytoene desaturase enzyme has an amino acid substitution corresponding to one or more of positions 288, 409, 493, and 526 of SEQ ID NO: 8.

30. The herbicide-resistant crop plant of claim 23, wherein the crop plant is soybean.

31. The herbicide-resistant crop plant of claim 30, wherein the soybean plant includes a polynucleotide encoding a modified soybean phytoene desaturase enzyme.

32. The herbicide-resistant crop plant of claim 25, wherein the modified soybean phytoene desaturase enzyme has an amino acid substitution corresponding to one or more of positions 294, 415, 499, and 532 of SEQ ID NO: 4.

33. A method for making an herbicide-resistant crop plant, comprising: modifying a crop plant to incorporate in its genome a polynucleotide containing a nucleic acid sequence encoding a modified plant phytoene desaturase enzyme having increased resistance to one or more bleaching herbicides, the modified plant phytoene desaturase enzyme having at least one amino acid substitution that provides said increased resistance.

34. A method according to claim 33, wherein said modifying comprises introducing said polynucleotide so as to form a transgenic, herbicide-resistant crop plant.

35. A method according to claim 33, wherein said modifying comprises modifying a native phytoene desaturase gene of the crop plant so as to form a non-transgenic, herbicide-resistant crop plant.

36. A method for controlling the growth of undesired vegetation growing at a location where a plant has been cultivated, said plant having an expressible nucleotide sequence encoding a plant phytoene desaturase protein having at least one point mutation relative to the wild-type nucleotide sequence encoding plant phytoene desaturase protein such that said plant is rendered resistant to a bleaching herbicide;
said method comprising applying to the location an effective amount of said bleaching herbicide.

37. The method of claim 36, wherein said expressible nucleotide sequence is selected from:
(a) a polynucleotide encoding a plant phytoene desaturase enzyme having an amino acid sequence at least 80% identical to amino acids 109 to 580 of SEQ ID NO: 2, said amino acid sequence having a point mutation corresponding to one or more of positions 304, 425, 509, and 542 of SEQ ID NO: 2;
(b) a polynucleotide encoding a plant phytoene desaturase enzyme having an amino acid sequence at least 80% identical to amino acids 97 to 570 of SEQ ID NO: 4, said amino acid sequence having a point mutation corresponding to one or more of positions 294, 415, 499, and 532 of SEQ ID NO: 4;
(c) a polynucleotide having encoding a plant phytoene desaturase enzyme having an amino acid sequence at least 80% identical to amino acids 97 to 571 of SEQ ID NO: 6, said amino acid sequence having a point mutation corresponding to one or more of positions 292, 413, 497 and 530 of SEQ ID NO: 6; and
(d) a polynucleotide having encoding a plant phytoene desaturase enzyme having an amino acid sequence at least 80% identical to amino acids 93 to 566 of SEQ ID NO: 8, said amino acid sequence having a point mutation corresponding to one or more of positions 288, 409, 493, and 526 of SEQ ID NO: 8.

38. The method of claim 37, wherein said plant is maize, soybean, or rice.

39. The method of claim 38, wherein said plant is maize.

40. The method of claim 39, wherein said expressible nucleotide sequence includes a polynucleotide having encoding a plant phytoene desaturase enzyme having an amino acid sequence at least 80% identical to amino acids 97 to 571 of SEQ ID NO: 6, said amino acid sequence having a point mutation corresponding to one or more of positions 292, 413, 497 and 530 of SEQ ID NO: 6.

41. The method of claim 38, wherein said plant is rice.

42. The method of claim 41, wherein said expressible nucleotide sequence includes a polynucleotide having encoding a plant phytoene desaturase enzyme having an amino acid sequence at least 80% identical to amino acids 93 to 566 of SEQ ID NO: 8, said amino acid sequence having a point mutation corresponding to one or more of positions 288, 409, 493, and 526 of SEQ ID NO: 8.

43. The method of claim 38, wherein said plant is soybean.

44. The method of claim 43, wherein said expressible nucleotide sequence includes a polynucleotide encoding a plant phytoene desaturase enzyme having an amino acid sequence at least 80% identical to amino acids 97 to 570 of SEQ ID NO: 4, said amino acid sequence having a point mutation corresponding to one or more of positions 294, 415, 499, and 532 of SEQ ID NO: 4.

45. A method for selecting for a bleaching herbicide resistant cell, tissue or plant, comprising providing within the cell, tissue or plant an expressible nucleotide sequence encoding a plant phytoene desaturase protein having at least one point mutation relative to the wild-type nucleotide sequence encoding plant phytoene desaturase protein, such that said plant is rendered resistant to a bleaching herbicide; and
applying to the cell, tissue or plant an effective amount of said bleaching herbicide.

46. A method according to claim 45, wherein said expressible nucleotide sequence is coupled to a second nucleotide sequence for providing a desired trait to be introduced into the cell, tissue or plant.

47. A method according to claim 46, wherein said providing includes introducing into the cell, tissue or plant a transformation vector containing the expressible nucleotide sequence and second nucleotide sequence.

48. A method according to any of claims 45-47, wherein said expressible nucleotide sequence is selected from:
(a) a polynucleotide encoding a plant phytoene desaturase enzyme having an amino acid sequence at least 80% identical to amino acids 109 to 580 of SEQ ID NO: 2, said amino acid sequence having a point mutation corresponding to one or more of positions 304, 425, 509, and 542 of SEQ ID NO: 2;
(b) a polynucleotide encoding a plant phytoene desaturase enzyme having an amino acid sequence at least 80% identical to amino acids 97 to 570 of SEQ ID NO: 4, said amino acid sequence having a point mutation corresponding to one or more of positions 294, 415, 499, and 532 of SEQ ID NO: 4;
(c) a polynucleotide having encoding a plant phytoene desaturase enzyme having an amino acid sequence at least 80% identical to amino acids 97 to 571 of SEQ ID NO: 6, said amino acid sequence having a point mutation corresponding to one or more of positions 292, 413, 497 and 530 of SEQ ID NO: 6; and
(d) a polynucleotide having encoding a plant phytoene desaturase enzyme having an amino acid sequence at least 80% identical to amino acids 93 to 566 of SEQ ID NO: 8, said amino acid sequence having a point mutation corresponding to one or more of positions 288, 409, 493, and 526 of SEQ ID NO: 8.

49. A method according to claim 48, wherein the cell, tissue or plant is a maize cell, maize tissue, or maize plant.

50. A method according to claim 48, wherein the cell, tissue or plant is a rice cell, rice tissue, or rice plant.

51. A method according to claim 48, wherein the cell, tissue or plant is a soybean cell, soybean tissue, or soybean plant.
